(19) United States
(12) Patent Application Publication
(10) Pub. No.: US 2009/0214637 A1
(43) Pub. Date: Aug. 27, 2009

(54) Novel Gene Therapy Approach For Treating The Metabolic Disorder Obesity

(75) Inventor: Sergey Musatov, New York, NY (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: NEUROLOGIX, INC., Fort Lee, NJ (US)

(21) Appl. No.: 12/433,098

(22) Filed: Apr. 30, 2009

Related U.S. Application Data
(63) Continuation-in-part of application No. 12/261,451, filed on Oct. 30, 2008.
(60) Provisional application No. 61/001,011, filed on Oct. 30, 2007.

(51) Int. Cl. A61K 9/127; A61K 31/7088; A61K 45/00; A61P 3/04

(52) U.S. Cl. 424/450; 424/93.6; 514/44 A

(57) ABSTRACT

The present application relates to novel methods and compositions for treating metabolic disorders. Some aspects pertain to the use of gene therapy to treat diseases related to metabolic disorders, such as diabetes, obesity, high blood pressure, wasting syndrome, cachexia and atherogenic dyslipidemia. The present application also pertains to the use of vectors such as a recombinant adeno-associated virus (AAV) to deliver a at least a portion of a gene that can increase or decrease expression of a therapeutic protein of interest, e.g., in cells in a specific region of the brain associated with metabolic disorder. The present application also discloses the use of vectors such as a recombinant adeno-associated virus for the delivery of small interference RNA's (siRNAs) capable of decreasing expression of a deleterious protein involved in the disorder. Other related aspects, including compositions related to such methods, are also disclosed.

### CROSS REFERENCE TO RELATED APPLICATIONS

The present application is a continuation in part application that claims the benefit of U.S. Provisional Application No. 61/001,011 filed on Oct. 30, 2007, and U.S. patent application Ser. No. 12/261,451 filed on Oct. 30, 2008, both entitled “A Novel Gene Therapy Approach For Treating The Metabolic Disorder Obesity.” The entire contents of both applications are hereby incorporated by reference.

### FIELD OF INVENTION

This invention relates to novel methods and compositions for treating metabolic disorders. More specifically, the invention pertains to the use of gene therapy to treat diseases related to metabolic dysfunction, such as diabetes, obesity, high blood pressure, and atherogenic dyslipidemia. The invention also pertains to the use of vectors to deliver a gene capable of increasing or decreasing expression of a therapeutic protein of interest in cells in a specific region of the brain associated with metabolic dysfunction.

### BACKGROUND

Disorders of metabolic pathways play an important role in the progression of various disease processes. For example, diseases such as type-II diabetes, hypertension, high cholesterol, atherogenic dyslipidemia have been identified as arising from disorders related to metabolism. The term “metabolic syndrome” has been coined to refer to a cluster of conditions that occur together, and increase the risk for heart disease, stroke and diabetes. Having just one of these conditions such as increased blood pressure, elevated insulin levels, excess body fat around the waist or abnormal cholesterol levels increases the risk of the above mentioned diseases. In combination, the risk for coronary heart disease, stroke and diabetes is even greater.

Research into the complex underlying processes linking this group of conditions is ongoing. As the name suggests, metabolic syndrome is tied to the body's metabolism, and more likely to a condition called insulin resistance. Although, not all experts agree on the definition of metabolic syndrome or whether it even exists as a distinct medical condition, this collection of risk factors is becoming prevalent with an estimated 50 million Americans suffering from some form of metabolic disorder.

Obesity is a chronic disease manifested by an excess of fat mass in proportion to body size. Today, every third American is considered over-weight (Body Mass Index (BMI) >25 kg/m²), thus prompting the United States Centers for Disease Control and Prevention (CDC) to declare that obesity is reaching epidemic proportions (Cummings et al., Genetics and Pathophysiology of Human Obesity, Annu. Rev. Med., vol. 54, pg. 453, 2003). The importance of treating obesity is emphasized by the fact that this disease is either the underlying cause, or a risk factor, for developing diseases such as type 2 diabetes, congestive heart failure, osteoarthritis and sleep apnea among others.

Obesity can also be linked to metabolic syndrome. As discussed above, metabolic syndrome can be characterized by excess body fat, atherogenic dyslipidemia, elevated blood pressure and insulin resistance. It has been shown, however, that even a modest decrease in body weight (5-10% of initial body weight) may significantly improve conditions associated with the metabolic syndrome and decrease the risk factors for developing obesity-associated disease (Tuomilehto et al., Prevention of type 2 diabetes mellitus by changes in lifestyle among subjects with impaired glucose tolerance, New Engl. J. Med., vol. 344, pg. 1343, 2001; Knowler et al., Reduction in the incidence of type 2 diabetes with lifestyle intervention or metformin, New Engl. J. Med., vol. 346, pg. 393, 2002; Franz et al., Evidence-based nutrition principles and recommendations for the treatment and prevention of diabetes and related complications, Diabetes Care, vol. 25, pg. 148, 2002). Additionally, treatment of obesity may be important from a mental health perspective due to the social stigma often attached to obese individuals in some cultures.

Obesity is caused by both genetic and environmental factors. Genetic causes of this abnormality can result from a single gene mutation in animals, but humans rarely develop obesity from a single gene mutation (Chaganon, et al., The Human Obesity Gene Map: The 1997 Update, Obes. Res. vol. 6, pg. 76, 1998). To develop treatments for obesity, studies delineating the pathophysiology of body weight regulation are vital. It has been found that fat serves not only as a reservoir for energy but also causes the secretion of substances involved in energy homeostasis. One such substance is “leptin” a hormone whose concentration in blood serum is related to the proportion of body fat. Leptin regulates body fat content and energy expenditure by influencing the brain. Mutations in the gene for leptin or its receptors have been identified as one possible cause for obesity. Leptin has also been implicated to be associated with hyperphagia, hyperinsulinemia, and insulin resistance (Prasad, et al., A Paradoxical Elevation of Brain Cyclo (his-pro) Levels in Hyperphagic Obese Zucker Rats, Brain Res. vol. 699, pg. 149, 1995). Initial studies focused on the use of leptin for treating obesity showed that common diet-induced obese mice were relatively insensitive to increased concentrations of systemic leptin. These studies also showed that the reduced sensitivity to systemically administered leptin arose either from a reduced transport of leptin to the brain, or due to inhibition of leptin-induced signal transduction to the hypothalamus. Thus, there is a need for therapy targeted towards the treatment of leptin-resistant obesity.

Obesity is currently treated, with only limited success, by several different strategies. These strategies primarily involve “life-style” changes (e.g. diet and exercise), small molecule based pharmaceutical therapies or surgical removal of a portion of the stomach (gastric by-pass surgery). Additionally, weight loss stimulating melanocortin receptor binding peptides such as alpha-MSH are of limited use as pharmaceuticals due to the extremely short serum half-life of such peptides. In addition, drug treatment for obesity has been disappointing since almost all drug treatments for obesity were associated with undesirable side effects that contribute to the termination of their prolonged use as therapeutics. Available pharmacotherapies have included Sibutramine, Orlistat, fenfluramine and dexfenfluramine. Fenfluramine and dexfenfluramine were withdrawn from the market in 1997 because of associated cardiac valvulopathy (Connolly et al., Valvular Heart Disease Associated With Fenfluramine-Phentermine, New Engl. J. Med., vol. 337, pg. 581, 1997). Therefore, health care professionals continue to be reluctant to use pharmacotherapy in the management of obesity. Complimentary approaches to pharmacotherapy are therefore of great interest to the public. In this application, we propose a novel gene therapy approach for the treatment of obesity.

### SUMMARY OF THE INVENTION

The present invention is drawn to methods for treating a metabolic disorder. In some embodiments, at least a portion of a gene can be provided to increase or decrease expression of a therapeutic protein of interest to at least one cell. A vector can be used for delivering the gene. Expression of the therapeutic protein is increased or decreased in the transfected cells thereby treating the metabolic disorder.

Accordingly, one embodiment of the instant invention relates to treatments for metabolic disorders such as obesity, type-2 diabetes, hypertension, wasting syndrome, cachexia and atherogenic dyslipidemia. As the name suggests, metabolic disorders are tied to the body's metabolism and can result in excessive weight gain and/or excessive weight loss. Excess body fat can lead to disorders such as obesity, type-2 diabetes, hypertension and atherogenic dyslipidemia. Conversely, disorders such as wasting syndrome and cachexia can lead to excessive weight loss.

Another embodiment of the invention relates to methods for silencing gene expression of a therapeutic protein by providing a polynucleotide sequence that functions as at least one of a shRNA, a siRNA and a RNAi. Preferably, the polynucleotide is homologous to at least a portion of an estrogen receptor-alpha gene (ERα) and decreases ERα protein expression to treat the metabolic disorder. The method can further include delivering a vector comprising the homologous ERα polynucleotide to a glucose-responsive neuron of a ventromedial nucleus (VMN). As shown in the examples, silencing ERα expression in the VMN lead to a significant gain in the body weights and overall obese phenotype in mice.

In one more embodiment, the method comprises altering expression of multiple genes. For example, multiple genes can be silenced. The method can comprise providing a second polynucleotide homologous to at least a portion of a second gene, such as huntingtin interacting protein 2 (Hip2), to decrease expression of a protein encoded the second gene by incorporating the polynucleotide into a vector, transfecting the vector into at least one neuron, such as in the hypothalamus and decreasing expression of the corresponding protein. Furthermore, multiple genes can be silenced with one vector, or the polynucleotides homologous to the genes can be on separate vectors to silence the multiple genes. In another example, expression of multiple genes can be increased. The method can comprise providing at least a portion of a second gene, such as Hip2, to increase expression of a protein encoded the second gene by incorporating the gene into a vector, transfecting the vector into at least one neuron, such as in the hypothalamus and increasing expression of the corresponding protein. Likewise, the vector can comprise multiple genes and/or multiple vectors can be used to increase expression of multiple genes.

In yet another embodiment, the therapeutic agent of the invention can be selected from the group consisting of huntingtin interacting protein 2 (Hip2), peroxisome proliferator-activated receptor γ coactivator 1α (PGC1-α), and estrogen receptor-alpha (ERα). In some particular embodiments, the therapeutic protein is an estrogen receptor-alpha (ERα). The gene incorporated into the vector can comprise at least a portion of a gene from at least one of huntingtin interacting protein 2 (Hip2), peroxisome proliferator-activated receptor γ coactivator 1α (PGC1-α), and estrogen receptor-alpha (ERα). In a preferred embodiment, the incorporated gene is at least a portion of an ERα gene to increase expression of at least a portion of an ERα protein.

Another embodiment of the invention provides incorporating the gene for use in a vector (e.g., viral or non-viral) to deliver the gene. A viral vector can be selected from a group consisting of adeno-associated viral vector, herpes simplex viral vector, parovirus vector and lentivirus vectors. In a preferred embodiment, the viral vector is an adeno-associated viral vector (AAV). Furthermore, the adeno-associated virions can have a cap-region from one type of AAV and a rep-region from a second type of AAV which is distinct from the first AAV. A particularly favorable adeno-associated virion has a non-native capsid from AAV-1 and a rep-region from AAV-2. Such recombinant AAV's have the advantage of exhibiting modified tropism, (i.e., being highly selective with respect to the tissues it infects), as well as having a higher rate of transduction efficiency when compared to native AAV. In another embodiment, the vector can be a non-viral vector, more specifically, it can be a liposome-mediated delivery vector.

In some embodiments, a vector is delivered to a desired region of the central nervous system using stereotaxic delivery. In a preferred embodiment, the vector is delivered to a desired region of a brain. It is further advantageous to deliver the vector within a region of the brain that is associated with a particular disorder. In a preferred embodiment, the region of the brain is selected from the group consisting of hypothalamus, ventromedial nucleus, and arcuate nucleus. Preferrably, the vector can be delivered to the ventromedial nucleus of a mammalian brain. More preferrably, the vector can be delivered to a glucose-responsive neuron of the ventromedial nucleus.

Another aspect of the instant invention pertains to the treatment of obesity by identifying a target site in a brain of a patient that requires modification, and transfecting at least one cell at the target site with a vector expressing a therapeutic protein, and followed by the expressing the therapeutic protein in an amount effective for modulating metabolism in the patient. The therapeutic protein can be selected from the group consisting of brain derived neurotrophic factor (BDNF), huntingtin interacting protein 2 (Hip2), peroxisome proliferator-activated receptor γ coactivator 1α (PGC1-α), estrogen receptor-alpha (ERα), glial neurotrophic factor (GNF), erythropoietin (EPO), granulocyte colony stimulating factor (G-CSF), thrombopoietin (TPO), growth hormone (GH), interleukin 2 (IL-2), interferon-alpha receptor, interferon-beta receptor, and insulin receptor. Alternatively, the expressed therapeutic protein is the estrogen receptor-alpha (ERα). Accordingly, the invention also relates to the treatment of obesity, by altering the basal metabolic rate in an obese subject so as to cause a reduction in body weight by expressing the therapeutic protein.

In another embodiment of the invention, the target site can be in the brain. More preferrably, the target site can consist of at least one of a hypothalamus, a ventromedial nucleus and an arcuate nucleus. The method can also comprise transfecting at least one cell of the brain, where the cell is a glucose-responsive neuron of a ventromedial nucleus. Methods are also disclosed for delivering the vector to these target sites. Preferrably, the vector can be delivered by at least one of an oral administration, a nasal administration, a buccal administration, an intravenous injection, an intra-peritoneal injection, an intrathecal administration, and a route appropriate for delivering the vector to a particular region of the brain.

In still another aspect of this invention, a pharmaceutical composition for treating a metabolic disorder is disclosed, where the metabolic disorders can be obesity, hypertension, diabetes, wasting syndrome, cachexia and athrogenic dyslipidemia. In a preferred embodiment the disorder is obesity. The pharmaceutical composition can comprise an effective amount of an adeno-associated viral vector encoding at least a portion of a gene to increase or decrease expression of a therapeutic protein in a desired region of a brain and a pharmaceutically acceptable carrier to treat the metabolic disorder.

The vector of the pharmaceutical composition can encode at least a portion of a gene, where the gene can encode therapeutic proteins such as huntingtin interacting protein 2 (Hip2), brain derived neurotropic factor (BDNF), peroxisome proliferator-activated receptor γ coactivator 1α (PGC1-α), and estrogen receptor-alpha (ERα). Preferably, the gene encoding the therapeutic protein can be at least a portion of an estrogen receptor-alpha (ERα) gene and the therapeutic protein can be at least a portion of the ERα protein.

The pharmaceutical composition can further increase or decrease expression of a second gene through the use of same vector or multiple vectors. A vector can comprise at least a portion of a Hip2 gene to increase expression of at least a portion of a Hip2 protein in a neuron of a hypothalamus. The vector can also comprise a polynucleotide sequence that functions as at least one of a shRNA, a siRNA and a RNAi to decrease expression of the therapeutic protein to therapeutically effective levels. Preferably, the polynucleotide sequence is homologous to at least a portion of an estrogen receptor-alpha (ERα) gene. The pharmaceutical composition can further comprise a vector comprising a polynucleotide sequence that is homologous to at least a portion of a second gene to decrease its expression. The second gene can be Hip2 to decrease expression of Hip2 protein in a neuron of a hypothalamus.

In one embodiment of the pharmaceutical composition, the vector can alter expression of the therapeutic protein in a desired region of the brain, where the region of the brain is at least one of a hypothalamus, a ventromedial nucleus, and an arcuate nucleus. Preferrably, the therapeutic protein is functional in a ventromedial nucleus of a mammalian brain. More preferrably, the therapeutic protein is functional in a glucose-responsive neuron of the ventromedial nucleus.

### BRIEF DESCRIPTION OF THE FIGURES

Embodiments of the invention can be more readily understood with reference to the following figures:

FIG. 1A reproduces a micrograph depicting expression of Hip2 in murine hypothalamus. Hip2 is widely expressed in the hypothalamus, with highest levels observed in the dorso-medial nucleus (DMN), the arcuate nucleus (ARC) and the ventro-medial nucleus (VMN).

FIG. 1B reproduces a micrograph depicting an assay for the detection of specific Hip2 small hairpin RNA. Suppression of Hip2 expression in brain can be performed using AAV vectors encoding for Hip2-specific small hairpin RNA (shRNA). Three different Hip2 shRNA target sequences were tested for their ability to silence the expression of the gene that codes for Hip2.

FIG. 2A depicts the effect of Hip2 silencing on body weight. Male mice were injected into the VMN with AAV vectors encoding for luciferase (Luc) or Hip2 shRNA. Animals from the two groups had similar body weights prior to surgery (week 0). However, mice with suppressed Hip2 levels in the VMN showed an increase in body weight shortly after vector injection.

FIG. 2B depicts the effect of Hip2 silencing on change in body weight of male mice. The figure graphs the change in body weight of male mice as a function of time in weeks post-administration of AAV vectors encoding for luciferase (Luc) or Hip2 shRNA.

FIG. 2C shows that Hip2 silencing increases the weight of gonadal fat pads in male mice. Graph of the change in the weight of gonadal fat pads of male mice after administration of AAV vectors encoding for luciferase (Luc) or Hip2 shRNA.

FIG. 3 shows the effect of Hip2 silencing on cold-induced hypothermia. Core body temperature was measured at ambient temperature (22° C.) and following exposure to cold (4° C.) for 4 h. The test was performed during the dark phase of the daily cycle with full access to food and water. The animals injected with Hip2 shRNA vector developed a more profound hypothermia suggesting an impaired response to acute cold stress.

FIG. 4 depicts the effect of Hip2 silencing on fasting-induced hypothermia. Animals were fasted for 24 h with full access to water and core temperature was measured 2 h after the onset of the dark phase and compared to the temperature of the same animals fed ad libitum (freely fed). The mice treated with Hip2 shRNA vector displayed reduced ability to maintain core body temperature following fast.

FIG. 5A depicts the effect of Hip2 silencing on diet-induced thermogenesis. Suppression of Hip2 levels in the VMN resulted in impaired thermogenic response following food consumption after a 24-h fast. The absolute values for body temperature were lower for Hip2 shRNA-treated mice.

FIG. 5B shows that the body temperature of fed mice injected with either Hip2 shRNA or Luc increases by 2C when compared to mice that have received AAV vectors expressing the same gene in the fasted group.

FIG. 6A-B depicts the effect of Hip2 silencing on daily food intake. No difference in daily food intake was observed between the two groups (A) at three weeks; (B) six weeks after surgery. At the time points noted, Hip2 shRNA-treated mice had already displayed a significant increase in body weight (FIG. 2). These results indicate that weight gain was not associated with increased consumption of food.

FIG. 7A-F shows that mice injected with AAV vectors encoding Hip2 shRNA showed similar levels of daily physical activity measured as total distance (A), distance dark phase (B), distance light phase (C), stereotypical counts (D), stereotypical dark phase (E) and sterotypical light phase (F) as control mice administered Luciferase specific shRNA.

FIG. 8A-B depicts the effect of Hip2 silencing on the response to 2-deoxy-D-glucose (2-DG) induced hypoglycemia. 2-DG significantly increased food intake over 4 h compared to saline, however, no difference was observed between the group receiving Luc shRNA and the group receiving Hip2 shRNA; (A) at 1 h following injection of 2-DG; and (B) at 4 h following injection of 2-DG.

FIG. 9A depicts the effect of estrogen receptor-alpha (ERα) on the expression of genes responsible for neuronal glucose sensing. Decreases were observed in the expression of glucose kinase (GK) in the VMN region of the mouse brain and in glucose-responsive neuronal N43 cells after administration of AAV vectors encoding ERα sh-RNAi versus control vectors encoding for luciferase activity.

FIG. 9B shows that decreases were observed in the expression of Kir6.2 in the VMN region of the mouse brain and in glucose-responsive neuronal N43 cells after administration of AAV vectors encoding ERα sh-RNAi versus control vectors encoding for luciferase.

FIG. 9C shows that similar levels of expression were seen for the lactose transporter MCT1 in the VMN region of the mouse brain and in glucose responsive neuronal N43 cells after administration of AAV vectors encoding ERα sh-RNAi versus control vectors encoding for luciferase.

FIG. 10 shows that mice were injected with indicated vectors and their body weight was monitored over a period of several weeks. At the end of the experiment, the animals were sacrificed and the accuracy of injections and the efficiency of ERα silencing were assessed using YFP and ERα immunostaining. Animals with unilateral (siER1-U) and bilateral (siER1-B) injections were analyzed separately. *p<0.05.

FIG. 11A shows that three weeks after surgery animals were fasted for 24 h with full access to water, injected with glucose (2 mg/kg, i.p.) and their blood glucose concentration was monitored for 2 h. The test was performed during the dark phase of the daily cycle. Higher levels of blood glucose following the challenge were noted in siER1-treated mice. *p<0.05.

FIG. 11B shows that three weeks after surgery were fasted for 24 h with full access to water, injected with glucose (2 mg/kg, i.p.) and their blood glucose concentration was monitored for 2 h. The test was performed during the dark phase of the daily cycle. Lower fasting glucose concentrations were noted in siER1-treated mice compared to control animals. *p<0.05.

FIG. 12A shows that core body temperature was measured during the glucose tolerance test described above. While siER1-treated mice increased their body temperature after glucose injection, both baseline and induced values were significantly lower compared to control animals. *p<0.05.

FIG. 12B shows that knockdown of ERα in the VMN reduced core body temperature in both fasted and ad libitum fed (freely fed) mice. *p<0.05.

FIG. 12C depicts the results of an experiment where animals were systemically injected with 2-deoxy-D-glucose (250 mg/kg, i.p.) and their body temperature was monitored for 2 h. Mice treated with siER1 vector developed a more profound and sustained hypothermia compared to control animals (*p<0.05).

FIG. 12D shows that animals injected with siER1 displayed reduced ability to maintain body temperature following an acute cold stress (4° C.). *p<0.05.

FIG. 13A-E shows that expression of glucokinase (A), Kir6.2 (B), GLUT3 (C), MCT1 (D) and GLUT4 (E) genes implicated in neuronal glucosensing was analyzed in dissected VMN regions by quantitative PCR. Suppression of ERα in the VMN significantly reduced mRNA levels of several genes including glucokinase, a pore-forming subunit of an ATP-dependent potassium channel, Kir6.2, and glucose transporter, GLUT3. *p<0.05.

FIG. 14A-E shows that glucose-responsive murine hypothalamic N43 cells were transduced with AAV vectors over-expressing ERα or YFP. Expression levels of glucokinase (A), Kir6.2 (B), GLUT3 (C), MCT1 (D) and GLUT4 (E) genes were analyzed 48 h later by quantitative PCR. *p<0.05.

FIG. 15A shows that glucose-excited neurons (GE) increased firing rate in 2.5 mM glucose and decreased firing rate in 0.5 mM glucose and express membrane ERα.

FIG. 15B shows that glucose-inhibited neurons (GI) decreased firing rate in 2.5 mM glucose and increased firing rate in 0.5 mM glucose and express membrane ERα.

FIG. 15C shows that high glucose-excited neurons (HGE) increased firing rate in 10 mM glucose and decrease firing rate in 2.5 mM glucose and express membrane ERα.

FIG. 15D shows that non glucose-sensing displayed no response to varying glucose concentrations and express nuclear ERα.

FIG. 16A shows that a majority of membrane ERα-immunoreactive cells (GE) depolarized following application of 1 nM or 10 nM 17β-estradiol.

FIG. 16B shows that cells with nuclear ERα staining did not depolarize following application of 1 nM or 10 nM 17β-estradiol.

FIG. 17 shows that the effect of estradiol on the number of glucose sensing VMN neurons in culture in culture was analyzed after treatment with estradiol.

FIG. 18A shows that after 4 days in culture, cultured neurons containing glucose-excited (GE) glucose-responsive neurons transduced with siERα displayed a lack of responsiveness to glucose, even over a wide range of concentrations, indicating membrane ERα is important for glucose sensing.

FIG. 18B shows that after 4 days in culture, cultured neurons containing glucose-inhibited (GI) glucose-responsive neurons transduced with siERα displayed a lack of responsiveness to glucose, even over a wide range of concentrations, indicating membrane ERα is important for glucose sensing.

FIG. 18C shows that shows that after 4 days in culture, cultured neurons containing high glucose-excited (HE) glucose-responsive neurons transduced with siERα displayed a lack of responsiveness to glucose, even over a wide range of concentrations, indicating membrane ERα is important for glucose sensing.

FIG. 18D shows that non-glucose sensing cells with nuclear ERα displayed no response to glucose when transduced with a vector targeting ERα (siERα).

### DETAILED DESCRIPTION OF THE INVENTION

The present application relates to novel methods and compositions for treating metabolic disorders. Some aspects pertain to the use of gene therapy to treat diseases related to metabolic disorders, such as diabetes, obesity, high blood pressure, wasting syndrome, cachexia and atherogenic dyslipidemia. The present application also pertains to the use of vectors such as a recombinant adeno-associated virus (AAV) to deliver a gene that can increase or decrease expression of a therapeutic protein of interest, e.g., in cells in a specific region of the brain associated with metabolic disorder. The present application also discloses the use of vectors such as a recombinant adeno-associated virus for the delivery of small interference RNA's (siRNAs) capable of decreasing expression of a deleterious protein involved in the disorder. Other related aspects, including compositions related to such methods, are also disclosed.

The term “metabolic syndrome” refers to a collection of factors such as central obesity, insulin resistance, hypertension, dyslipidemia, and chronic inflammation that increase the risk of individuals to diseases associated with metabolic syndrome. Particularly susceptible individuals are those who have a poor diet and nutrition, those who lead sedentary life-styles, as well as individuals with a genetic pre-disposition to diseases associated with metabolic disorders.

In some embodiments, the therapeutic potential of huntingtin interacting protein 2 (Hip2), estrogen receptor alpha (ERα) and/or neurotrophic growth factors in the treatment of metabolic disorders are utilized. For instance, Hip2 is a ubiquitin conjugating enzyme expressed in the brain. Recent evidence, from experiments involving obese mice, has implicated that this protein has a crucial role in energy homeostasis and body weight regulation. It appears that, physiologically, Hip2 exerts control over metabolism and appears to be essential for maintaining the normal energy balance in cells. Thus, gene therapy strategies aimed at altering the steady state concentration of Hip2 provide an attractive route for treating obesity.

Thus in at least one aspect the invention is directed towards the use of vectors to deliver the gene for Hip2 to the brain of a subject to treat obesity. In a preferred embodiment, the vector carrying the gene for Hip2 is delivered to the hypothalamus of the subject.

In addition to gene therapy directed towards the modulation of Hip2 levels in the hypothalamus, a second aim of the instant invention is the use of gene therapy to alter proteins that are downstream to Hip2 in the signaling cascade and may be involved in energy homeostasis. Recent studies have identified and focused on the peroxisome proliferator-activated receptor γ coactivator 1α protein (PGC1-α), which is a key protein implicated to play a role in mitochondrial biogenesis and respiration. Studies on transcriptional regulation of energy metabolism have suggested that PGC1-α is a co-activator of the nuclear receptor PPAR-γ that regulates fat development. Thus, gene therapy approaches aimed at modulating the activity of PCP1-α could potentially find applications as therapeutics for the treatment of metabolic disorders. Alternatively, PGC1-α may pose as an attractive target in the treatment of diabetes since this protein is involved in controlling hepatic gluconeogenesis.

Growth factors, such as brain derived neurotrophic factor (BDNF), play an important role in signaling, energy metabolism, and in the overall control of body weight regulation. In particular, the ventromedial nucleus (VMN) of the hypothalamus is the key center involved in BDNF controlled energy homeostasis. In fact, there is significant support in the literature for the involvement of brain derived neurotrophic factor in body weight regulation and activity.

Furthermore, there appears to be a direct correlation between the steady state concentration of BDNF in the brain in relation to the concentration of systemic leptin. In many obese people the leptin-controlled signaling cascade responsible for decreasing food intake in response to increasing concentration of leptin is either inefficient or non-functional. Whatever the reason for the malfunctioning of this signaling process, the observation, that an increase in systemic leptin leads to increases in BDNF expression in the brain, provides motivation for gene therapy targeting concentrations of central BDNF, and provides an attractive strategy for the treatment of leptin-resistant obesity. Thus, the invention provides methods and compositions for gene therapy aimed at treating feeding disorders and obesity by increasing the amount of BDNF centrally, or by modulating the concentration of its high affinity receptor.

Another factor that has been demonstrated to play an important role in regulation of food intake and energy expenditure is the estrogen receptor α (ERα). Recent studies have shown that ERα modulates the glucose sensing function of the glucose responsive (GR) neurons in the ventromedial nucleus (VMN) region of the hypothalamus, and that a disruption of ERα signaling can lead to an obese phenotype and the development of disorders related to the metabolic syndrome.

These results suggest a role for ERα in determining glucose levels in the hypothalamus. Furthermore, the results suggest that a method for treating obesity and other diseases linked to metabolic syndromes can be by delivering a gene capable of expressing ERα to the VMN neurons, so as to increase the in-vivo steady state level of this receptor as well as the sensitivity of the cells in the brain to changes in glucose concentrations. The current invention discloses methods and compositions for delivering the gene for ERα using AAV vectors or non-viral delivery methods.

Thus, some aspects of the current application are directed towards the use of vectors such as AAV vectors to deliver the gene for ERα into the VMN neurons, so as to improve the ability of these neurons to sense blood glucose levels and thus modulate energy homeostasis.

Some aspects of the present invention employ, conventional techniques of virology, microbiology, molecular biology, and recombinant DNA techniques within the skill of the art. Such techniques are fully explained in the literature. (See, e.g., Sambrook, et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M. Knipe, eds.)).

### I. DEFINITIONS

So that the present application is more clearly understood, the following terms are defined. Various terms can also/alternatively, when appropriate, be delineated as typically understood by one skilled in the art.

The term “central nervous system” or “CNS” pertains to the brain, cranial nerves and spinal cord. The CNS also comprises the cerebrospinal fluid, which fills the ventricles of the brain and the central canal of the spinal cord.

The term “hypothalamus” pertains to a portion of the brain that contains a number of small nuclei with a variety of functions. One of the most important functions of the hypothalamus is to link the nervous system to the endocrine system via the pituitary gland. The hypothalamus is located below the thalamus, just above the brain stem. The hypothalamus is responsible for certain metabolic processes and other activities of the autonomic nervous system. It synthesizes and secretes neurohormones that in turn stimulate or inhibit the secretion of the pituitary hormones.

The terms “ventromedial nucleus” and “VMN” pertain to a nucleus of the hypothalamus. The VMN is a medially located nucleus of the hypothalamus that is situated between the lateral wall of the third ventricle and the formix and that is held to suppress the urge to eat when satiety is reached. Neurons of the VMN can be glucose-responsive (GR) or non-responsive. In addition, the glucose-responsive neurons can be further divided into glucose-excited (GE), glucose-inhibited (GI), high glucose-excited (HGE) neurons.

As used herein, the term “metabolic disorders” refers to medical conditions characterized by problems with an organism's metabolism. Since a healthy, functioning metabolism is crucial for life, metabolic disorders are treated very seriously. A broad range of conditions including, but not limited to, diabetes (including hyperglycemia and hypoglycemia), hyper/hypo-thyroidism, wasting syndrome, cachexia and obesity are some examples of disorders that can be classified as metabolic disorders. Metabolic disorders can result in excessive weight gain and/or excessive weight loss. The term “metabolic syndrome” refers to a cluster of conditions that occur together, and increase the risk for heart disease, stroke and diabetes. Having just one of these conditions such as increased blood pressure, elevated insulin levels, excess body fat around the waist or abnormal cholesterol levels increases the risk of the above mentioned diseases. In combination, the risk for coronary heart disease, stroke and diabetes is even greater. The main features of metabolic syndrome include insulin resistance, hypertension, cholesterol abnormalities, and an increased risk for clotting. Patients are most often overweight or obese.

As used herein, the term “polypeptide” refers to a single amino acid or a polymer of amino acid residues. A polypeptide may be composed of two or more polypeptide chains. A polypeptide includes a protein, a peptide, an oligopeptide, and an amino acid. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention, e.g., formation of a disulfide bond, glycosylation, lipidation, methylation, acetylation, phosphorylation, or by manipulation, such as conjugation with a labeling component.

As used herein, the term “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

As used herein, the term “specifically binding,” refers to the interaction between binding pairs (e.g., an antibody and an antigen). In various instances, specifically binding can be embodied by an affinity constant of at most 10⁻⁶ moles/liter, at most 10⁻⁷ moles/liter, or at most 10⁻⁸ moles/liter.

As used herein, the terms “RNAi,” “siRNA,” “shRNA,” refer to a RNA polynucleotides, small interfering RNA or short hairpin RNA, respectively. RNAi, siRNA and shRNA are used in various methods of RNA interference for gene silencing, as described in more detail below. The terms also refer to a single or double stranded RNA strand of any length, wherein at least one strand, putatively the antisense strand, is homologous to the target mRNA to be degraded. The difference between antisense and double stranded small interfering molecules is that an antisense molecule is a single stranded oligonucleotide which is complementary to a section of the target RNA and must hybridize or bind to it in a 1:1 ratio in order to cause its degradation. In contrast, siRNA provides a substrate for the RNA-induced silencing complex (RISC), and unlike antisense, is inactive until incorporated into this macromolecular complex. This RISC complex is then guided by the unwound siRNA to its target gene. Once the target gene is located, it is destroyed by cleaving the target gene into small pieces, and thereby preventing its expression.

In a preferred embodiment, the siRNA of the present invention comprises a double-stranded RNA duplex of at least about 15, or preferably at least about 19, nucleotides with no overhanging nucleotides. In another embodiment, the siRNA of the present invention has nucleotide overhangs. For example, the siRNA may have two nucleotide overhangs, thus the siRNA will comprise a 21 nucleotide sense strand and a 21 nucleotide antisense strand paired so as to have a 19 nucleotide duplex region. The number of nucleotides in the overhang can be in the range of about 1 to about 6 homologous nucleotide overhangs at each of the 5′ and 3′ ends, preferably, about 2-4, more preferably, about 3 homologous nucleotide overhangs at each of the 5′ and 3′ ends. The nucleotides overhang can be modified, for example to increase nuclease resistance. For example, the 3′ overhang can comprise 2′ deoxynucleotides, e.g., TT, for improved nuclease resistance.

The term “homology” or “identity” as used herein refers to the percentage of likeness between nucleic acid molecules. To determine the homology or percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 95%, 99% or 99.5% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The term “subject” refers to any living organism. The term subject comprises, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered. In preferred embodiments, the subject is a mammal, including humans and non-human mammals. In a more preferred embodiment, the subject is a mammal. In the most preferred embodiment, the subject is a human.

The term “tissue-specific” refers to regulatory sequences that can preferentially direct expression of the gene in a particular cell type, i.e., tissue-specific regulatory elements can be used. One skilled in the art would be familiar with tissue-specific regulatory elements or promotors for a specific target site or target cell type. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Preferred promoters are those functional in the central nervous system. Particularly preferred promoters are Chicken beta Active (CBA) and neuron-specific elonase (NSE). The promoter can be any desired promoter, selected based on the level of expression required of the gene operably linked to the promoter and the cell type in which the vector is used.

### II. METABOLIC DISORDERS

### Obesity

Obesity is a disease that affects many Americans. The number of overweight and obese Americans has continued to increase since 1960, a trend that is not slowing down. Today, 64.5 percent of adult Americans (about 127 million) are categorized as being overweight or obese. Each year, obesity causes at least 300,000 excess deaths in the U.S., and healthcare costs of American adults with obesity amount to approximately $100 billion.

Obesity is a chronic disease with a strong familial component. Obesity increases one's risk of developing conditions such as high blood pressure, diabetes (type 2), heart disease, stroke, gallbladder disease and cancer of the breast, prostate and colon. The tendency toward obesity is fostered by our environment: lack of physical activity combined with high-calorie, low-cost foods.

Obesity is manifested by an excess of fat mass in proportion to body size. Today, every third American is considered over-weight (Body Mass Index (BMI) >25 kg/m²), thus prompting the United States Centers for Disease Control and Prevention (CDC) to declare that obesity is reaching epidemic proportions (Cummings and Schwartz, Annu. Rev. Med. 54:453-471((2003)).

Obesity is caused by both genetic and environmental factors. To develop treatments for obesity, studies delineating the pathophysiology of body weight regulation are vital. It has been found that fat serves not only as a reservoir for energy but also causes the secretion of substances involved in energy homeostasis. One such substance is “leptin” a hormone whose concentration in blood serum is related to the proportion of body fat. Leptin regulates body fat content and energy expenditure by influencing the brain. Mutations in the gene for leptin or its receptors have been identified as one possible cause for obesity.

Obesity is currently treated, with only limited success, by several different strategies. These strategies primarily involve “life-style” changes (e.g. diet and exercise), small molecule based pharmaceutical therapies or surgical removal of a portion of the stomach (gastric by-pass surgery). In addition, drug treatments for obesity have been disappointing since most drugs used for treating obesity are associated with undesirable side effects that contribute to the termination of their use. Therefore, health care professionals continue to be reluctant to use pharmacotherapy in the management of obesity. Complimentary approaches to pharmacotherapy are therefore of great interest to the public.

### Diabetes

Type 2 diabetes is the most common form of diabetes. In type 2 diabetes, either the body does not produce enough insulin or the cells ignore the insulin. Insulin is necessary for the body to be able to use glucose. Cells use glucose as energy, and insulin is responsible for allowing glucose to enter cells. People with impaired metabolism have an increased propensity to develop type 2 diabetes. Diabetes has been linked to the metabolic syndrome. Particularly, recent studies have shown that ERα in the VMN region of the hypothalamus plays an important role in glucose sensing as well as in energy homeostasis. It has been shown that an under expression of the ERα protein or its absence results in obesity and an increase in body weight. Thus, delivery of viral vectors encompassing a gene capable of expressing ERα in the VMN region could provide an alternative approach to diabetes therapy.

Hypoglycemia is the clinical syndrome that results from low blood sugar. The symptoms of hypoglycemia can vary from person to person, as can the severity but principal problems arise from an inadequate supply of glucose as fuel to the brain resulting in impairment of function. Classically, hypoglycemia is diagnosed by a low blood sugar with symptoms that resolve when the sugar level returns to the normal range. Hypoglycemia occurs when blood glucose drops below normal levels. While patients who do not have any metabolic problems can complain of symptoms suggestive of low blood sugar, true hypoglycemia usually occurs in patients being treated for diabetes (type 1 and type 2). Patients with pre-diabetes who have insulin resistance can also have low blood sugars on occasion if their high circulating insulin levels are further challenged by a prolonged period of fasting. There are other rare causes for hypoglycemia, such as insulin producing tumors (insulinomas) and certain medications. These uncommon causes of hypoglycemia will not be discussed in this article, which will primarily focus on the hypoglycemia occurring with diabetes mellitus and its treatment.

### Wasting Syndrome

Wasting is sometimes referred to as “acute malnutrition” because it is believed that episodes of wasting have a short duration. A common problem among HIV-infected people is the HIV wasting syndrome, defined as unintended and progressive weight loss often accompanied by weakness, fever, nutritional deficiencies and diarrhea. The syndrome, also known as cachexia, can diminish the quality of life, exacerbate illness and increase the risk of death for people with HIV.

Wasting can occur as a result of HIV infection itself but also is commonly associated with HIV-related opportunistic infections and cancers. Most patients with advanced HIV disease and AIDS eventually experience some degree of wasting. Many approaches have been used to reverse weight loss in HIV-infected people, including appetite stimulants, anabolic agents, cytokine inhibitors and testerone hormone therapy. Goals of therapy include both increase in body weight and increase in lean body mass (muscle).

Currently, the precise causes of the HIV wasting syndrome are not well known, and probably vary among individuals. However, a growing body of evidence suggests that many factors may contribute to wasting including inadequate dietary intake, malabsorption of nutrients, abnormalities in metabolism and energy expenditure, and HIV-related infections. The mechanism may involve cachectin—also called tumor necrosis factor, a macrophage-secreted cytokine.

### Cachexia

Cachexia is loss of weight, muscle atrophy, fatigue, weakness and significant loss of appetite in someone who is not actively trying to lose weight. It can be a sign of various underlying disorders; when a patient presents with cachexia, a doctor will generally consider the possibility of cancer, metabolic acidosis (from decreased protein synthesis and increased protein catabolism), certain infectious diseases (e.g. tuberculosis, AIDS), and some autoimmune disorders, or addiction to drugs such as amphetamines or cocaine. Cachexia physically weakens patients to a state of immobility stemming from loss of appetite, asthenia and anemia, and response to standard treatment is usually poor.

While the primary cause of cachexia is not anorexia or decreased caloric intake, this complex metabolic disorder involves increased tissue catabolism. Protein synthesis is decreased and degradation increased. Cachexia is mediated by certain cytokines, especially tumor necrosis factor-α, IL-1b, and IL-6, which are produced by tumor cells and host cells in the tissue mass. The ATP-ubiquitin-protease pathway plays a role as well. However, additional caloric supplementation does not relieve cachexia. Any weight gain is usually minimal and is likely to consist of adipose tissue rather than muscle. Thus, in most cachectic patients with cancer, high-calorie supplementation is not recommended, and parenteral nutritional support is not indicated, except in situations where oral intake of adequate nutrition is impossible. However, other treatments can mitigate cachexia and improve function. Corticosteroids can increase appetite and may improve a sense of well-being but do little to increase body weight

### III. GENES

### Hip2

Huntingtin interacting protein 2 or Hip2 (also known as E2-25K) (NM_{—}016786) (SEQ ID NO.: 1) encodes a protein (NP_{—}058066) (SEQ ID NO.: 2) that belongs to the ubiquitin-conjugating enzyme family and is a member of the E2 protein family that catalyzes multiubiquitin chain synthesis via Lys48 of ubiquitin. It has been reportedly involved in Alzheimer's disease, Huntington's disease and antigen processing through its interaction with amyloid-β, huntingtin, and MHC-heavy chain proteins. It binds selectively to a large region at the N terminus of huntingtin and has been implicated in the degradation of huntingtin and suppression of apotosis. Hip2 may also mediate ubiquitination of the neuronal intranuclear inclusions in Huntington disease, thereby modulating aggregation and toxicity of expanded huntingtin protein. In addition, Lys14 of Hip2 can be modified by SUMOylation, with this modification resulting in inhibited E2 activity.

Recent evidence, from experiments involving obese mice, has implicated that Hip2 has a crucial role in energy homeostasis and body weight regulation. For example, preliminary evidence suggests that there is a direct correlation between Hip2 levels in the hypothalamus and sensitivity to diet-induced obesity in mice. Mice injected with an AAV vector encompassing the si-RNA to the gene for Hip2, showed an increase in body weight. Interestingly, the increase in body weight was due to decrease in the basal metabolic level of these animals, rather than an increase in their food intake. It therefore appears that, physiologically, Hip2 exerts control over metabolism and appears to be essential for maintaining the normal energy balance in cells.

In addition to gene therapy directed towards the modulation of Hip2 levels in the hypothalamus, a second aim of the instant invention is the use of gene therapy to alter proteins that are downstream to Hip2 in the signaling cascade and may be involved in energy homeostasis. Recent studies have identified and focused on the protein PGC1-α, which is a key protein implicated to play a role in mitochondrial biogenesis and respiration. Studies on transcriptional regulation of energy metabolism have suggested that PGC1-α is a co-activator of the nuclear receptor PPAR-γ that regulates fat development. Interestingly, it was found that biological control of critical metabolic processes does not occur via the changes in the amounts and activities of key transcriptional factors as was originally thought, but rather occurs via transcriptional co-activation processes. For example, it has been shown that PGC1-α, co-activates both brown fat mediated thermogenesis as well as hepatic gluconeogenesis. Furthermore, PGC1-α also initiates β-oxidation of fatty acids in the liver. Thus, gene therapy approaches aimed at modulating the activity of PCP1-α could potentially find applications as therapeutics for the treatment of obesity. Alternatively, PGC1-α may pose as an attractive target in the treatment of diabetes since this protein is involved in controlling hepatic gluconeogenesis.

Growth factors, such as brain derived neurotrophic factor (BDNF), play an important role in signaling, energy metabolism, and in the overall control of body weight regulation. In particular, the ventromedial nucleus (VMN) of the hypothalamus is the key center involved in BDNF controlled energy homeostasis. In fact, there is significant support in the literature for the involvement of brain derived neurotrophic factor in body weight regulation and activity. For example, heterozygous BDNF knockout mice (Bdnf(+/−)) are hyperphagic, obese, and hyperactive; furthermore, central infusion of BDNF leads to severe, dose-dependent appetite suppression and weight loss in rats, (Friedel S, et al., Mutation screen of the brain derived neurotrophic factor gene (BDNF): Identification of several genetic variants and association studies in patients with obesity, eating disorders, and attention-deficit/hyperactivity disorder, Am. J. Med. Genet. B Neuropsychiatr. Genet. vol. 132, pg. 96, 2005). Further support for the role of BDNF in feeding behavior arises from the observation that mice with reduced expression of BDNF or its high affinity receptor, TrkB, exhibit hyperphagia and obesity. Finally, there appears to be a direct correlation between the steady state concentration of BDNF in the brain in relation to the concentration of systemic leptin.

In many obese people the leptin-controlled signaling cascade responsible for decreasing food intake in response to increasing concentration of leptin is either inefficient or non-functional. One reason for this malfunction could be due to mutations in the leptin receptor, mutations in leptin itself or the inability of the hormone to reach the desired regions of the brain. Whatever the reason for the malfunctioning of this signaling process, the observation, that an increase in systemic leptin leads to increases in BDNF expression in the brain, provides motivation for gene therapy targeting concentrations of central BDNF, and provides an attractive strategy for the treatment of leptin-resistant obesity. Thus, the invention provides methods and compositions for gene therapy aimed at treating feeding disorders and obesity by increasing the amount of BDNF centrally, or by modulating the concentration of its high affinity receptor.

### ERα

Estrogen receptor refers to a group of receptors that are activated by estrogen. Two types of estrogen receptor exist: nuclear hormone family of intracellular receptors and estrogen G-protein coupled receptor. The hormone activated receptor is formed by homo and heterodimers of α (NM_{—}007956) (SEQ ID NO.: 3) (NP_{—}031982) (SEQ ID NO.: 4) and β chains, each coded by separate genes. The estrogen receptor alpha and beta show significant overall sequence homology, and both are composed of seven domains. Due to alternative RNA splicing, several ER isoforms are known to exist. At least three ERα and five ERβ isoforms have been identified.

ERα stimulates transcription of target genes by means of two distinct activation functions, AF1 in the N-terminal domain and AF2 in the ligand binding domain, whose activities vary depending upon the target promoter and cell type. The activity of AF1 is ligand independent and constitutive but can be modulated by phosphorylation by the mitogen activated protein kinase (MAPK) pathway in response to growth factors (Kato S. et al., Oncology 55 (suppl 1), 5-10. 1998). ERα and ERβ exhibit similar but not identical ligand binding properties. Both receptors appear to contain a functionally conserved AF2 which depends on the binding of estradiol. Although the two receptors are poorly conserved in the N-terminal domain, ERβ, like ERα, appears to contain a MAPK phosphorylation site that results in enhanced transcriptional activity. However, the activity of AF1 in ERβ is negligible compared with that of ERα on estrogen responsive element (ERE) based promoters. As a consequence, when transcription from a gene depends on both AF1 and AF2, the activity of ERα greatly exceeds that of ERβ, but when AF1 is not required, ERα and ERβ have similar transcriptional activities.

The presence of ER-alpha has been investigated in a number of widely used breast cancer cell (BCC) lines and its presence in breast cancer patients correlates with a lower risk of relapse and better overall survival. Aging and metabolism related studies in female mice have shown that ERα declines in the pre-optic hypothalamus, region responsible for thermoregulation, as they grow older when placed on a non restricted diet. However female counterparts on a caloric-restricted diet, maintained higher levels of ERα in the pre-optic hypothalamus.

ERα protein that has been implicated in regulating food intake and energy expenditure. For example, ERα knock-out mice have been shown to develop several hallmark features associated with obesity, including increased visceral adiposity, elevated insulin levels and a reduced glucose tolerance. Recent studies have indicated that ERα plays a role in glucose sensing by modulation of the glucose-responsive (GR) neurons in the VMN. GR neurons use protein glucose kinase (GK) as a key regulator for sensing glucose levels. GK modulates potassium (K)-ATP channel activity via a control over the production of ATP during glycolysis. Studies using si-RNA to silence ERα have shown that ERα silencing is accompanied by a concomitant decrease in the expression of glucose kinase and a second protein Kir6.2 which is a subunit of the K-ATP channel. Additional studies have indicated that estrogen receptor-alpha (ERα) in the ventromedial nucleus (VMN) region of the hypothalamus is important for controlling body weight and energy homeostasis in the presence of estrogens as disruption of ERα signaling can lead to an obese phenotype and the development of disorders related to the metabolic syndrome (Musatov et al., Silencing of estrogen receptor alpha in the ventromedial nucleus of hypothalamus leads to metabolic syndrome, PNAS, vol. 104, pg. 2501, 2007). Musatov et al., have shown that si-RNA's designed to silence ERα, when delivered to the VMN of mice using adeno-associated virus (AAV) vectors cause significant gain in the body weights of these animals versus control mice injected with AAV vectors encompassing luciferase. Furthermore, these researchers were able to show that the increase in body weight was not due to an increase in food intake by the animals. Rather, they concluded that the resultant increase in body weights was most likely due to a decrease in the basal metabolic activity of the animals.

Further studies have shown that ERα is expressed in glucose sensing neurons and modulates their function. Analysis of estrogen and glucose signaling pathways, displayed changes in neuronal activity induced by glucose in dissociated VMN neurons. It was also determined that the majority of glucose-excited neurons immunolabeled for ERα were located at the plasma membrane, unlike cells with nuclear ERα staining, becoming depolarized following application of 1 nM or 10 nM 17β-estradiol.

One embodiment of the invention can be directed to gene therapy strategies aimed at altering, i.e. increasing or decreasing, the expression of genes like Hip2 and/or ERα in the hypothalamus to treat metabolic disorders. Decreasing or inhibiting expression of genes like Hip2 and/or ERα can be accomplished by methods known to those skilled in the art, such as RNA interference techniques to silence genes like Hip2 and/or ERα.

Increasing genes like Hip2 and/or ERα can also provide a therapy to those with metabolic disorders that result in weight loss, such as wasting syndromes and cachexia. Gene therapy methods to increase expression or improve expression of genes like Hip2 and/or ERα can also be accomplished by methods known to those skilled in the art. For example, experimental results suggest that delivering a gene capable of expressing ERα to the VMN neurons, so as to increase the in-vivo steady state level of this receptor as well as the sensitivity of the cells in the brain to changes in glucose concentrations.

Another embodiment to the invention is directed to altering multiple genes, such as Hip2 and/or ERα, for a combined therapy to treat metabolic disorders. This can be accomplished through the use of one or more vectors for expression of at least a portion of the genes. The genes can be present within the same vector, allowing for the use of one vector capable of expressing both genes and delivered to a target site. The genes can also be in separate vectors. The use of separate vectors can allow for delivery to different target sites. The use of separate vectors can also allow for different expression constructs. For example, vectors with at least a portion of the Hip2 and/or ERα genes can be delivered to neurons of the hypothalamus and to neurons in the VMN of the hypothalamus, respectively.

The gene or genes can be an endogenous gene in relation to the cell, as in the case of a regulatory gene or a gene coding for a native protein, or it can be heterologous in relation to the cell, as in the case of a viral or bacterial gene, transposon, or transgene. The gene can also comprise a portion of the gene and encode at least 15 amino acids. Preferrably, the portion of the gene encodes at least 20, 30, 40, 50, 60, 75, 100 or more amino acids. More preferrably, the gene comprises the full length gene and encodes the entire protein. To alter expression of the protein, the cell is contacted with the gene in an amount sufficient to alter expression of the target gene, e.g., Hip2 and/or ERα.

### IV. GENE SILENCING THROUGH RNA INTERFERENCE

RNA interference (RNAi) is a mechanism that inhibits gene expression at the stage of translation or by hindering the transcription of specific genes. Small interfering RNA strands (siRNA) are key to the RNAi process, and have complementary nucleotide sequences to the targeted RNA strand. Small hairpin RNA or short hairpin RNA (shRNA) are similar to siRNA in that the sequences are complementary to the nucleotide sequences of the targeted RNA strand, however the RNA makes a tight hairpin turn that can be used to silence gene expression via RNA interference. The shRNA hairpin structure is cleaved by the cellular machinery into siRNA which is then bound to the RNA-induced silencing complex (RISC). This complex, in turn, binds to and cleaves mRNAs which match the siRNA breaking the mRNAs down into smaller portions that can no longer be translated into protein, allowing for robust gene specific suppression.

Methods for use of RNAi, including siRNA and shRNA, and construction of RNAi vectors are known by those skilled in the art and are discussed by Engelke and Rossi in Methods in Enzymology, Volume 392: RNA Interference, 1^{st} Edition, Elsevier Academic Press, San Diego, 2005, which is herein incorporated by reference in its entirety.

In a preferred embodiment, the siRNA of the present invention comprises a single or double-stranded RNA of at least about 15, or preferably at least about 19. In another embodiment, the siRNA of the present invention forms a double-stranded duplex and can have nucleotide overhangs. For example, the siRNA can have two nucleotide overhangs, thus the siRNA will comprise a 21 nucleotide sense strand and a 21 nucleotide antisense strand paired so as to have a 19 nucleotide duplex region. The number of nucleotides in the overhang can be in the range of about 1 to about 6 homologous nucleotide overhangs at each of the 5′ and 3′ ends, preferably, about 2-4, more preferably, about 3 homologous nucleotide overhangs at each of the 5′ and 3′ ends. The nucleotides overhang can be modified, for example to increase nuclease resistance. For example, the 3′ overhang can comprise 2′ deoxynucleotides, e.g., TT, for improved nuclease resistance.

The difference between antisense and double stranded small interfering molecules is that antisense molecule is a single stranded oligonucleotide which is complementary to a section of the target RNA and must hybridize or bind to it in a 1:1 ratio in order to cause it's degradation. In contrast, siRNA provides a substrate for the RNA-induced silencing complex (RISC), and unlike antisense, is inactive until incorporated into this macromolecular complex.

The term “complement” refers to a nucleotide sequence which is complementary to an indicated sequence and which is able to hybridize to the indicated sequences.

Also included within the present invention are sequence variants of the polynucleic acids as selected from any of the nucleotide sequences as given in any of the given SEQ ID numbers with sequence variants containing either deletion and/or insertions of one or more nucleotides, especially insertions or deletions of 1 or more codons, mainly at the extremities of oligonucleotides (either 3′ or 5′), or substitutions of some non-essential nucleotides by others (including modified nucleotides an/or inosine). Other preferred variant polynucleic acids of the present invention include sequences which are redundant as a result of the degeneracy of the genetic code.

Particularly preferred variant polynucleic acids of the present invention include also sequences which hybridize under stringent conditions with any of the polynucleic acid sequences of the present invention. Particularly, sequences which show a high degree of homology (similarity) to any of the polynucleic acids of the invention as described above. Particularly sequences which are at least 80%, 85%, 90%, 95%, 99%, 99.5% or more homologous to said polynucleic acid sequences of the invention. Preferably said sequences will have less than 20%, 15%, 10%, or 5% variation of the original nucleotides of said polynucleic acid sequence.

Polynucleic acid sequences according to the present invention which are homologous to the sequences as represented by a SEQ ID NO can be characterized and isolated according to any of the techniques known in the art, such as amplification by means of sequence-specific primers, hybridization with sequence-specific probes under more or less stringent conditions, serological screening methods or via the LiPA typing system.

The comparison of sequences and determination of percent identity between two sequences can be accomplished using a mathematical algorithm. For example, the percent identity between two amino acid sequences can be determined using the Needleman and Wunsch ((1970) J. Mol. Biol. (48):444-453) algorithm which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6. In another example, the percent identity between two nucleotide sequences is determined using the GAP program in the GCG software package, using a NWSgapdna. CMP matrix and a gap weight of 40, 50, 60, 70, or 80 and a length weight of 1, 2, 3, 4, 5, or 6. In yet another example, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty.

The term “inhibit” or “inhibiting” as used herein refers to a measurable reduction of expression of a target gene or a target protein. The term also refers to a measurable reduction in the activity of a target protein. Preferably a reduction in expression is at least about 10%. More preferably the reduction of expression is about 20%, 30%, 40%, 50%, 60%, 80%, 90% and even more preferably, about 100%.

In the present invention, siRNA are introduced into the cell rather than large dsRNA molecules, thus circumventing the initiation step of the mechanism. Although composed of two structural elements that resemble oligonucleotides used in antisense gene inhibition, the siRNA molecule has clear structural distinctions from the former. A siRNA molecule is composed of two complementary strands of RNA that must be hybridized with one another. There must be base-pair overhangs at each end of the molecule. Although the two oligonucleotides used for siRNA are the same length as those used for antisense, they will not be incorporated into the RISC complex unless they form this RNA duplex.

Where the siRNA of the present invention is delivered to a cell for the purposes of inhibiting expression of a target gene within the cell, at least one strand of the small interfering RNA is homologous to a portion of mRNA transcribed from the target gene, e.g. Hip2 and/or ERα. In a preferred embodiment, the siRNA strand is at least 85% homologous to a portion of mRNA transcribed from the target gene. Preferably, the siRNA strand is 90% homologous, more preferably is 95% homologous, and even more preferably, is 98% and 99% homologous to a portion of mRNA transcribed from the target gene, e.g., Hip2 and/or ERα. In the most preferred embodiment, at least one strand of the siRNA is 100% homologous to a portion of mRNA transcribed from the target gene, e.g., Hip2 and/or ERα.

In one embodiment, at least one siRNA molecule can be delivered to the cell, for example an siRNA molecule associated with a region of the gene, e.g., Hip2 and/or ERα. In another embodiment, a plurality of siRNA molecules can be delivered to the cell, for example, a plurality of siRNA molecules associated with one region of the Hip2 and/or ERα genes. In another embodiment, the plurality of siRNA molecules can be associated with different regions of the Hip2 and/or ERα genes. Thus, it will be appreciated that the scope of the invention covers any combination of siRNA molecules that can target and interfere with one or more desired regions of the Hip2 and/or ERα genes.

### IV. VECTORS

Some embodiments of the present invention utilize vectors that can be delivered to the cells of the central nervous system by using viral vectors or by using non-viral vectors. Preferred embodiments of the invention can use adeno-associated viral vectors comprising a nucleotide sequence encoding a chimeric receptor for gene delivery. AAV vectors can be constructed using known techniques to provide at least the operatively linked components of control elements including a transcriptional initiation region, an exogenous nucleic acid molecule, a transcriptional termination region and at least one post-transcriptional regulatory sequence. The control elements of the vector can be selected to be functional in the targeted cell. The resulting construct which contains the operatively linked components can be flanked at the 5′ and 3′ region with functional AAV inverted terminal repeats (ITR) sequences.

The nucleotide sequences of AAV ITR regions are known. The ITR sequences for AAV-2 are described, for example by Kotin et al. (1994) Human Gene Therapy 5:793-801; Berns “Parvoviridae and their Replication” in Fundamental Virology, 2nd Edition, Raven Press, N.Y., 1990. The skilled artisan will appreciate that AAV ITR's can be modified using standard molecular biology techniques. Accordingly, AAV ITRs used in the vectors of the invention need not have a wild-type nucleotide sequence, and can be altered, e.g., by insertion, deletion or substitution of nucleotides. Additionally, AAV ITRs can be derived from any of several AAV serotypes, including but not limited to, AAV-1, AAV-2, AAV-3, AAV-4, AAV-5, AAV-6, AAVX7, AAV-8 and the like. Furthermore, 5′ and 3′ ITRs which flank a selected nucleotide sequence in an AAV expression vector need not be identical or derived from the same AAV serotype or isolate, so long as the ITR's function as intended, i.e., to allow for excision and replication of the nucleotide sequence of interest when AAV rep gene products are present in the cell.

The skilled artisan can appreciate that regulatory sequences can often be provided from commonly used promoters derived from viruses such as: polyoma, Adenovirus 2, cytomegalovirus and Simian Virus 40. Use of viral regulatory elements to direct expression of the protein can allow for high level constitutive expression of the protein in a variety of host cells. Ubiquitously expressing promoters can also be used and include, for example, the early cytomegalovirus promoter as described by Boshart et al., Cell, vol. 41, pg. 521, 1985; herpesvirus thymidine kinase (HSV-TK) promoter as described by McKnight et al., Cell, vol. 37, pg. 253, 1984; β-actin promoters, e.g., the human β-actin promoter as described by Ng et al., Mol Cell Biol., vol. 5, pg. 2720, 1985; and colony stimulating factor-1 (CSF-1) promoter and described by Ladner et al., EMBO J., vol. 6, pg. 2693, 1987.

The AAV vector harboring the nucleotide sequence encoding a protein of interest, e.g., chimeric growth factor receptor, and a post-transcriptional regulatory sequence (PRE) flanked by AAV ITRs, can be constructed by directly inserting the nucleotide sequence encoding the protein of interest and the PRE into an AAV genome which has had the major AAV open reading frames (“ORFs”) excised therefrom. Other portions of the AAV genome can also be deleted, as long as a sufficient portion of the ITRs remain to allow for replication and packaging functions. These constructs can be designed using techniques well known in the art. (See, e.g., Lebkowski et al. (1988) Molec. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling et al. (1994) Gene Therapy 1:165-169; and Zhou et al. (1994) J. Exp. Med. 179:1867-1875).

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., Molecular Cloning a laboratory manual, Cold Spring Harbor Laboratories, N.Y., 1989. Several AAV vectors are available from the American Type Culture Collection (“ATCC”).

In order to produce recombinant AAV particles, an AAV vector can be introduced into a suitable host cell using known techniques, such as by transfection. A number of transfection techniques are generally known in the art. See, e.g.; Sambrook et al., Molecular Cloning a laboratory manual, Cold Spring Harbor Laboratories, N.Y., 1989; Davis et al., Basic Methods in Molecular Biology, Elsevier, San Diego, 1986. Particularly suitable transfection methods include calcium phosphate co-precipitation (Graham et al., Virology, vol. 52, pg. 456, 1973), direct micro-injection into cultured cells (Capecchi, Cell, vol. 22, pg. 479, 1980), electroporation (Shigekawa et al., BioTechniques, vol. 6, pg. 742, 1988), liposome mediated gene transfer (Mannino et al., BioTechniques, vol. 6, pg. 682, 1988), lipid-mediated transduction (Felgner et al., Proc. Natl. Acad. Sci. USA, vol. 84, pg. 7413, 1987), and nucleic acid delivery using high-velocity microprojectiles (Klein et al., Nature, vol. 327, pg. 70, 1987).

Suitable host cells for producing recombinant AAV particles include, but are not limited to, microorganisms, yeast cells, insect cells, and mammalian cells, that can be, or have been, used as recipients of a exogenous nucleic acid molecule.

Host cells containing the above-described AAV vectors must be rendered capable of providing AAV helper functions in order to replicate and encapsidate the expression cassette flanked by the AAV ITRs to produce recombinant AAV particles. AAV helper functions are generally AAV-derived coding sequences which can be expressed to provide AAV gene products that, in turn, function in trans for productive AAV replication. AAV helper functions are used herein to complement necessary AAV functions that are missing from the AAV vectors. Thus, AAV helper functions include one, or both of the major AAV open reading frames (ORFs), namely the rep and cap coding regions, or functional homologues thereof.

Alternatively, a vector can be a virus other than the adeno-associated virus, or portion thereof, which allows for expression of a nucleic acid molecule introduced into the viral nucleic acid. For example, replication defective retroviruses, adenoviruses, herpes simplex virus, and lentivirus can be used. Protocols for producing recombinant retroviruses and for infecting cells in vitro or in vivo with such viruses can be found described by Ausubel et al. in Current Protocols in Molecular Biology, Greene Publishing Associates, 1989, Sections 9.10-9.14 and other standard laboratory manuals. Examples of suitable retroviruses include pLJ, pZIP, pWE and pEM which are well known to those skilled in the art. Examples of suitable packaging virus lines include Crip, Cre, 2 and Am. The genome of adenovirus can be manipulated such that it encodes and expresses the protein of interest but is inactivated in terms of its ability to replicate in a normal lytic viral life cycle. See e.g., Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art.

Alternatively, the vector can be delivered using a non-viral delivery system. This includes delivery of the vector to the desired tissues in colloidal dispersion systems that include, for example, macromolecule complexes, nanocapsules, microspheres, beads, and lipid-based systems including oil-in-water emulsions, micelles, mixed micelles, and liposomes.

Liposomes are artificial membrane vesicles which are useful as delivery vehicles in vitro and in vivo. In order for a liposome to be an efficient gene transfer vehicle, the following characteristics should be present: (1) encapsulation of the genetic material at high efficiency while not compromising the biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al. (1988) Biotechniques, 6:682). Examples of suitable lipid liposome production include phosphatidyl compounds, such as phosphatidylglycerol, phosphatidylcholine, phosphatidylserine, phosphatidylethanolamine, sphingolipids, cerebrosides, and gangliosides. Additional examples of lipids include, but are not limited to, polylysine, protamine, sulfate and 3b-[N— (N′,N′ dimethylaminoethane) carbamoyl] cholesterol.

The cell receiving the vector of the present invention can be isolated, within a tissue, or within an organism. It can be an animal cell, a plant cell, a fungal cell, a protozoan, or a bacterium. An animal cell can be derived from vertebrates or invertebrates, but in a preferred embodiment of the invention, the cell is derived from a mammal, such as a rodent or a primate, and even more preferably, is derived from a human. The cell can be of any type, including neural cells, neuronal cells, epithelial cells, endothelial cells, muscle cells or nerve cells. Representative cell types include, but are not limited to, VMN neurons, microglia, myoblasts, fibroblasts, astrocytes, neurons, oligodendrocytes, macrophages, myotubes, lymphocytes, NIH3T3 cells, PC12 cells, and neuroblastoma cells. Such delivery may be accomplished either in vitro or in vivo by standard techniques.

### V. PHARMACEUTICAL COMPOSITIONS AND PHARMACEUTICAL ADMINISTRATION

The vector or the synthetic dimerizer used with some embodiments of the present invention can be incorporated into pharmaceutical compositions suitable for administration to a subject. In some particular embodiments, the pharmaceutical composition comprises the vector of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it can be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the vector or pharmaceutical composition.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular, intrathecal). In one embodiment, the vector is administered by intravenous infusion or injection. In another embodiment, the vector is administered by intramuscular or subcutaneous injection. In another embodiment, the vector is administered perorally. In the most preferred embodiment, the vector is delivered to a specific location using stereostatic delivery.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., vector) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be achieved by including an agent in the composition that delays absorption, for example, monostearate salts and gelatin.

Suitable pharmaceutical carriers, excipients and/or diluents include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water.

Where delivery is made in vivo to a living organism, administration can be by any procedure known in the art, including but not limited to, oral, parenteral, intraspinal, intracisternal, subdural, rectal, intradermal, transdermal, intramuscular, or topical administration. To facilitate delivery, the vector can be formulated in various compositions with a pharmaceutically acceptable carrier, excipient or diluent. “Pharmaceutically acceptable” means the carrier, excipient or diluent of choice does not adversely affect the biological activity of the dsRNA, or the recipient of the composition.

For oral administration, the composition may be presented as capsules or tablets, powders, granules or a suspension. The composition may be further presented in convenient unit dosage form, and may be prepared using a controlled-release formulation, buffering agents and/or enteric coatings.

The vector of the present invention can be administered by a variety of methods known in the art. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems by J. R. Robinson, ed., Marcel Dekker, Inc., New York, 1978.

One example of a delivery method involves delivering naked DNA molecules directly into the central nervous system of the subject. This can be accomplished by using a ventricular Omaya reservoir spinal catheter (e.g., portacath). Alternatively, cirect delivery of the siRNA molecules can be accomplished by using continuous spinal infusion using pump technologies (e.g., for Medtronic pump). For continuous spinal infusion, the lumbar catheterization protocol can be conducted by initially preparing a catheter using for example, polyethylene tubing (PE10) with outer diameter of about 0.6 mm, and a total tubing length of about 4.5 cm. A thin tungsten wire (e.g., with a diameter of about 0.12 mm) can be inserted into the PE10 tube as a guide wire. One end of the tubing can be stretched so the outer diameter shrinks. A triple knot can be made with silk suture at each end of the tubing in order to provide anchor points for the tubing after catheter implantation. An ALZET pump can be filled and primed with at least one siRNA molecule formulated in a delivery vehicle such as saline, dextrose, artificial cerebrospinal fluid, and the like. The siRNA can be delivered at a rate of about 6 μl/day. It will be appreciated that the volume of the siRNA formulation, and the rate at which it is delivered will depend on the size and weight of the subject. An adapter tube can be made using 0.69 mmID tubing cut to approximately 5 mm.

To implant the catheter, the subject, e.g., mice can be anesthetized with ketamine/domitor combination IP injection. The mice can be injected with Buprenex as a pain medication. A 2 cm longitudinal skin incision can be made above vertebrae L5 and L6. While holding the mouse's pelvic girdle firmly, a hole can be made in the muscle at the L5 and L6 junction using a 23 gauge needle. The needle can be gently pressed and spun through the muscle tissue. The catheter with metal wire inside can be pushed into the side of the L5-6 process initially at a 70 degree angle from the vertebral column. The angle can be flattened once resistance is reached until the catheter and wire is about 20-30 degrees from the vertebral column. The catheter with the wire can be pushed through the intervertebral space and dura until the sign of dura penetration (tail flick and/or hind limb quiver) occurs. At this point the guide wire is withdrawn in order to protect the spinal cord from damage. The catheter is then fed into the vertebral space until the silk suture knot rests adjacent to the hole in the muscle. A knot is tied through the fascia that rests superficially to the lumbar muscle so that the knot anchors the original silk catheter knot into its place. This keeps the catheter in place. The ALZET pump is attached to the catheter tubing using an adhesive and adaptor tube. The pump is implanted in the skin pocket. The second silk knot is anchored to the fascia at the neck with a suture knot. The incision is closed and the mice are dosed with Antesedan in order to counteract the Domitor.

The pharmaceutical compositions of the invention can include a “therapeutically effective amount” or a “prophylactically effective amount” of the vectors of the invention. A “therapeutically effective amount” refers to an amount effective, at dosages and for periods of time necessary, to achieve the desired therapeutic result. A therapeutically effective amount of the vector can vary according to factors such as the disease state, age, sex, and weight of the individual, and the ability of the vector to elicit a desired response in the individual. A therapeutically effective amount can also be one in which any toxic or detrimental effects of the vector are outweighed by the therapeutically beneficial effects. A “prophylactically effective amount” refers to an amount effective, at dosages and for periods of time necessary, to achieve the desired prophylactic result. Typically, since a prophylactic dose can be used in subjects prior to or at an earlier stage of disease, the prophylactically effective amount can be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It can be especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention can be dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Some aspects of the current invention are directed towards the use of vectors, such as AAV vectors, that can deliver at least a portion of a gene, such as the gene for ERα, to a cell such as a mammalian brain cell, either in vitro or in vivo. In some embodiments, the vectors can be delivered to a region of the brain, such as the hypothalamus, ventromedial nucleus (VMN), and arcuate nucleus. As disclosed herein, experiments have been conducted in mice where suppression of ERα by shRNAs led to an increase in body weight, decreased glucose tolerance, reduced body temperature and reduced expression of key glucosensing genes, such as glucose kinase and Kir6.2, a pore-forming subunit of the potassium ATP channel. As well, other experiments documented the overexpression of ERα in glucose-responsive hypothalamic N43 cells, which led to increased expression of glucose kinase (GK) and Kir6.2.

While not being bound by any particular theory, it is believed that the increased expression of GK can result in a desired therapeutic outcome. Cells of the ventromedial nucleus have been shown to sense blood glucose levels and thus modulate energy homeostasis. Glucose responsive neurons use protein GK as a regulator for sensing glucose levels. GK modulates potassium (K)-ATP channel activity via a control over the production of ATP during glycolysis. Increased levels of GK, as demonstrated by Niswender et al., J. Biol. Chem., vol. 272, pg. 22564, 1997; and Niswender et al., J. Biol. Chem., vol. 272, pg. 22570, 1997, herein incorporated by reference in their entirety, have been shown to result in increased glycogen synthesis, decreased body weight and increased metabolism in murine models. Accordingly, the present application supports the notion that ERα plays a significant role in determining glucose levels in the brain (e.g., hypothalamus), specifically the VMN and that increasing ERα expression levels leads to an increase in GK expression and function (as demonstrated by Kir6.2 increased expression). Overexpression of ERα in murine models demonstrated that increased GK led to increased metabolism and decreased body weight. Thus, increasing expression of ERα in the brain of a mammal, specifically the VMN, can increase GK expression and lead to a decrease in body weight.

Furthermore, the results suggest that a method for treating metabolic disorders can be by delivering a gene capable of expressing Hip2 to the hypothalamus and/or ERα to the VMN neurons, so as to increase expression of the gene as well as the sensitivity of the cells in the brain to changes in glucose concentrations. Thus, the current invention discloses methods directed to treating metabolic disorders, such as obesity, by transfecting at least one mammalian cell with a vector, such as an AAV vector, expressing a therapeutic protein, like ERα, to increase or decrease the level of the therapeutic protein, thereby treating the disorder. or non-viral delivery methods.

One skilled in the art can appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### EXAMPLES

### Example 1

### Effect of Hip2 Silencing on Body Weight of Mice

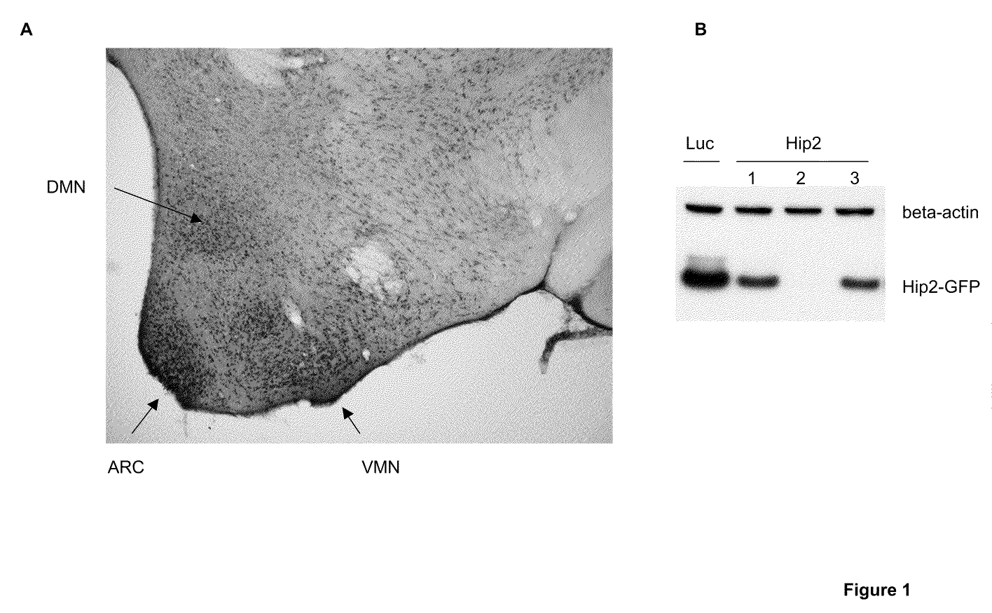

Prior to experimentation, Hip2 expression was characterized in murine hypothalamus. FIG. 1 shows the wide expression pattern of Hip2, with highest levels observed in dorso-medial nucleus (DMN), the arcuate nucleus (ARC) and the ventro-medial nucleus (VMN).

Male mice were injected with AAV vectors encoding Hip2 specific small hairpin RNA's (shRNA). AAV vectors encoding luciferase specific shRNA's were used as controls. The vectors were delivered to the ventromedial nucleus (VMN) region of the brain. After several weeks of monitoring the mice, the animals were sacrificed and the level of Hip2 suppression was assessed. Brain tissue from the mice injected with AAV vectors encoding for three different Hip2-specific shRNAs or a luciferase control were analyzed by PCR for the ability to silence Hip2 gene expression. Of the three, one shRNA was able to drastically reduce the level of Hip2 present in the brain, as measured by PCR.

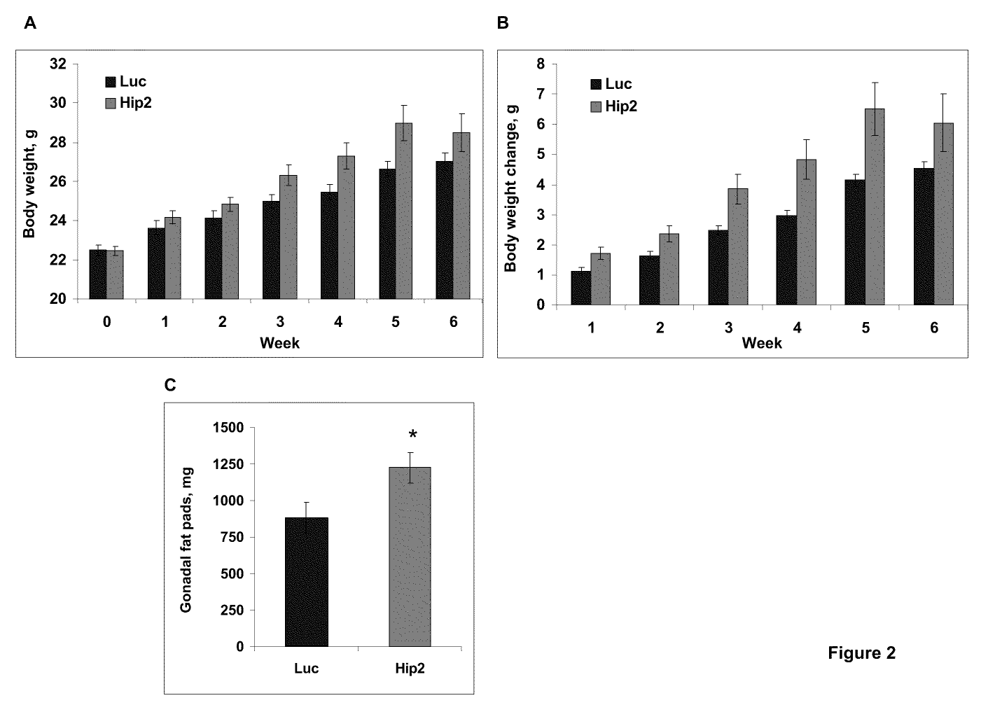

To determine the effect Hip2 silencing has on the mice, body weights were measured as an additional parameter. The body weights of mice in each group were determined prior to surgery and were found to be similar. Subsequently, the body weights of the animals in the two groups were measured each week following administration of AAV vectors for up to six weeks. The results are graphically depicted in FIG. 2A. The graph shows that the body weights of mice from the group that received an injection of AAV vectors encoding for Hip2 shRNA are greater than the body weights of mice that received injections of the control AAV vectors. The increase in the body weights of mice receiving Hip2 shRNAs was observed as early as one week post administration of the AAV vectors encoding for Hip2 shRNAs. However, the difference in body weights between the two groups was greater for weeks four, five, and six following surgery. FIG. 2B shows the overall change in body weights of mice within each group by comparing the weight of animals at weeks 1-6 post injection of the AAV vectors encoding either Hip2 shRNA or Luc shRNA, to the body weights of animals prior to surgery. Silencing of Hip2 expression resulted in a greater increase in the body weights of animals versus animals from the control group (Luc shRNA). Furthermore, it was noted that the increase in body weight resulted in increased accumulation of visceral fat, seen in FIG. 2C. The accumulation of visceral fat tissue was greater for animals in the Hip2 group than control animals.

### Example 2

### Effect of Hip2 Silencing on Cold-Induced Hypothermia

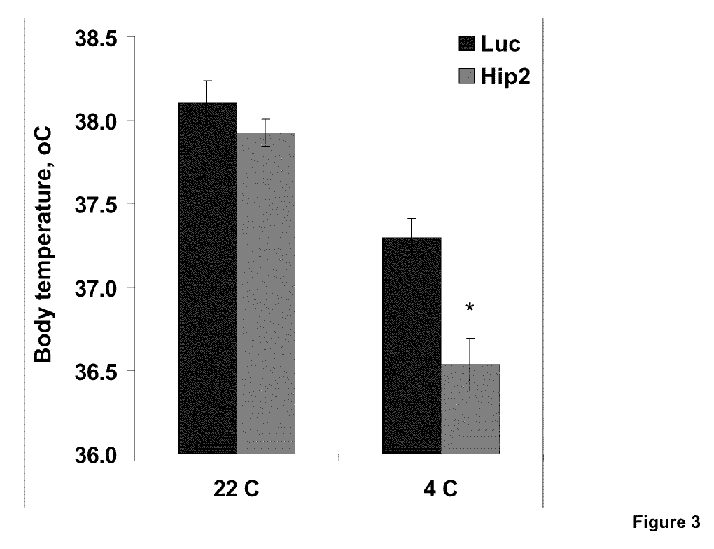

Male mice were divided into two groups, one group was injected with AAV vectors encoding for Hip2 shRNA while the other group was injected with AAV vectors encoding Luc shRNA. Mice were separated in cages according to group and placed in a room maintained at an ambient temperature of 22° C., or in cages placed in a room at 4° C. Mice were permitted access to food and water ad libitum (freely available), and measurements of core body temperature were made during the dark phase of the daily cycles. After 4 h at either 22° C., or at 4° C., the core body temperatures of the animals were recorded. The results are shown in FIG. 3. Animals maintained at 4° C. had lower core body temperatures than animals maintained at 22° C. At either temperature, the animals that had received injections of Hip2 shRNA had lower core body temperatures than animals in the control group. However, the core body temperatures of mice receiving Hip2 shRNA were much lower for animals kept at 4° C. than for animals kept at an ambient temperature of 22° C. These results indicate that Hip2 silencing impairs the animals' ability to respond to stress due to cold.

### Example 3

### Effect of Hip2 Silencing on Fasting-Induced Hypothermia

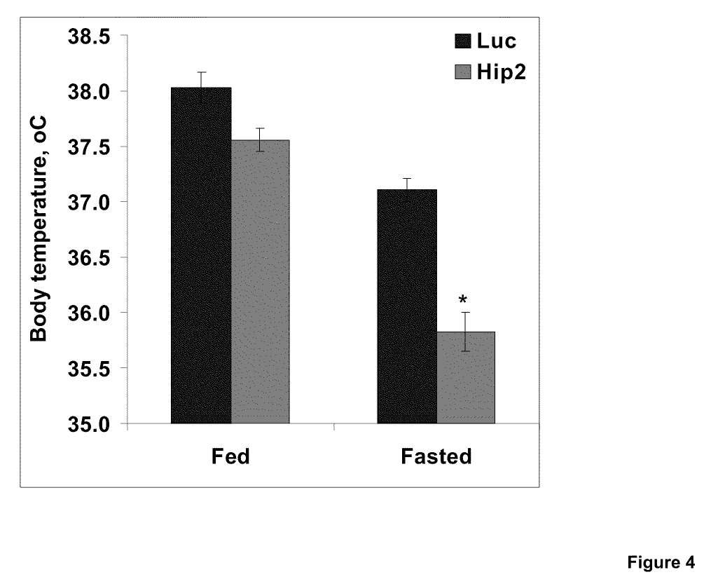

Male mice were divided into two groups, one group was injected with AAV vectors encoding for Hip2 shRNA while the other group was injected with AAV vectors encoding Luc shRNA. Mice from each group were either fasted for 24 h or allowed access to food ad libitum. For both the fasted and fed mice, access to water was allowed ad libitum. The measurements of core body temperature were made 2 h after the onset of the dark phase of the daily cycles. The results are shown in FIG. 4. The core body temperature was lower for animals that had been injected with the Hip2 shRNA versus control animals in both the fasted and fed groups. However, this effect was more pronounced for the animals that had fasted for 24 h prior to the measurements. This indicates that animals treated with Hip2 shRNA have a reduced ability to maintain core body temperature following fasting.

### Example 4

### Effect of Hip2 Silencing on Diet-Induced Thermogenesis

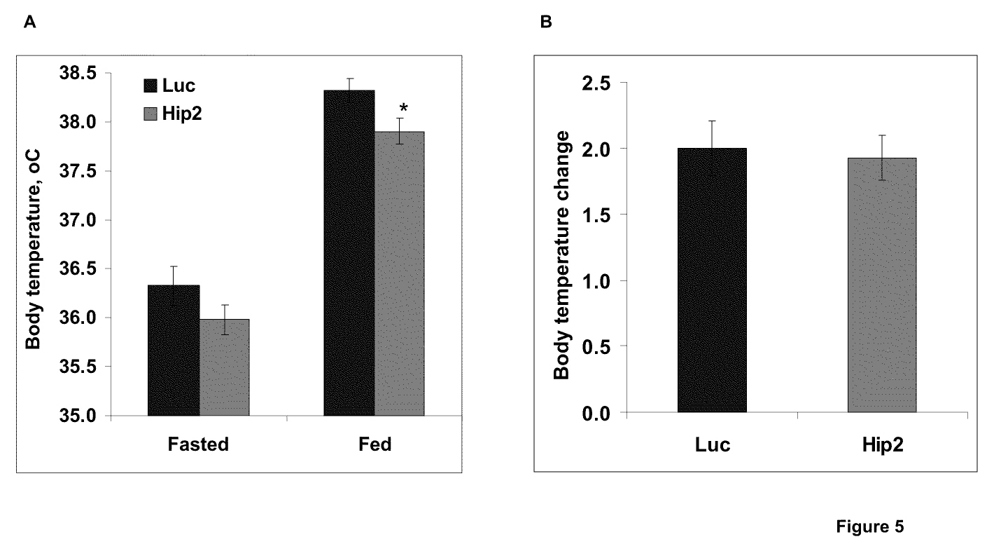

Male mice were divided into two groups, one group was injected with AAV vectors encoding for Hip2 shRNA while the other group was injected with AAV vectors encoding Luc shRNA. Mice from each group were either fasted for 24 h or allowed access to food ad libitum. Mice in both the fasted and fed groups were allowed access to water ad libitum FIG. 5A shows that the body temperature of fasted mice was lower than the body temperature of animals that were allowed to feed ad libitum. However, the overall difference in body temperature for mice injected with Hip2 shRNA in both the fasted and fed group was approximately 2° C., similar to the overall difference in body temperatures (FIG. 5B) for mice in the fed and fasted groups that received an injection of Luc shRNA.

Hip2 has been implicated to play a role in metabolism and in energy homeostasis making Hip2 an attractive target for therapeutic intervention, especially for the treatment of diseases associated with the metabolic syndrome. Results from various in vivo experiments have shown that suppression of endogenous Hip2 in mice resulted in an increase in the body weight of these animals. The increase in body weight was most often accompanied by an increase in the visceral adiposity in animals, which strongly correlated with several hallmark features of diseases associated with metabolic syndrome.

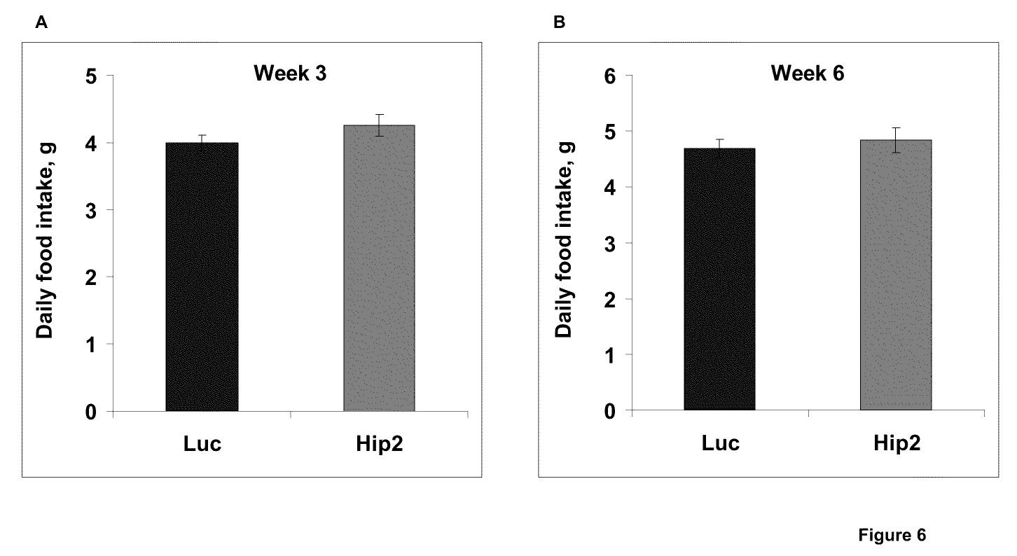

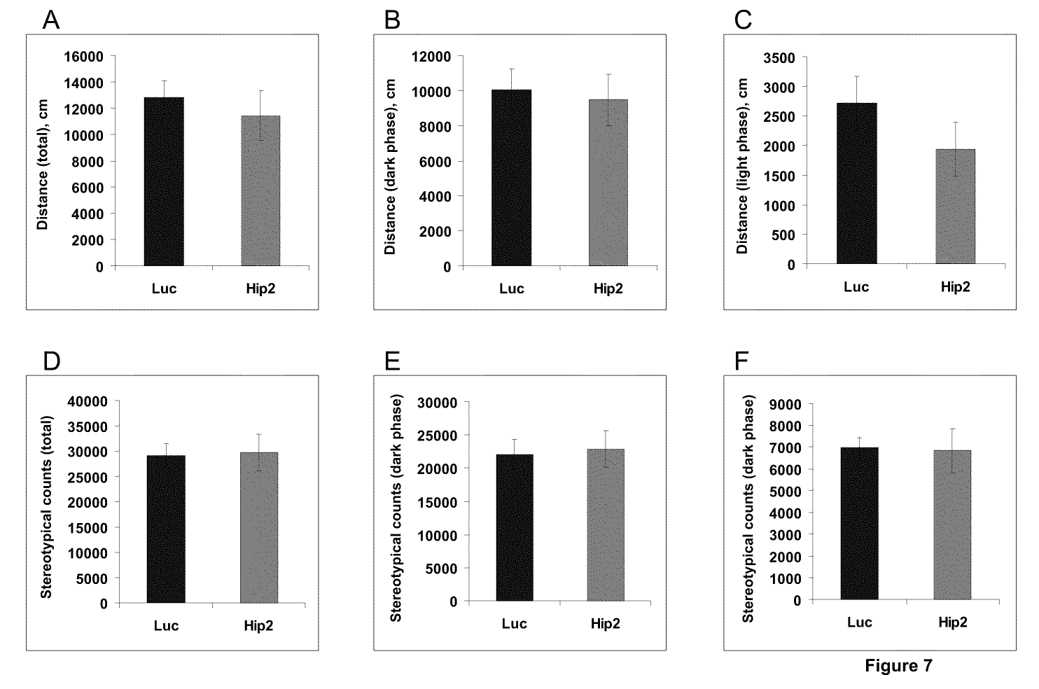

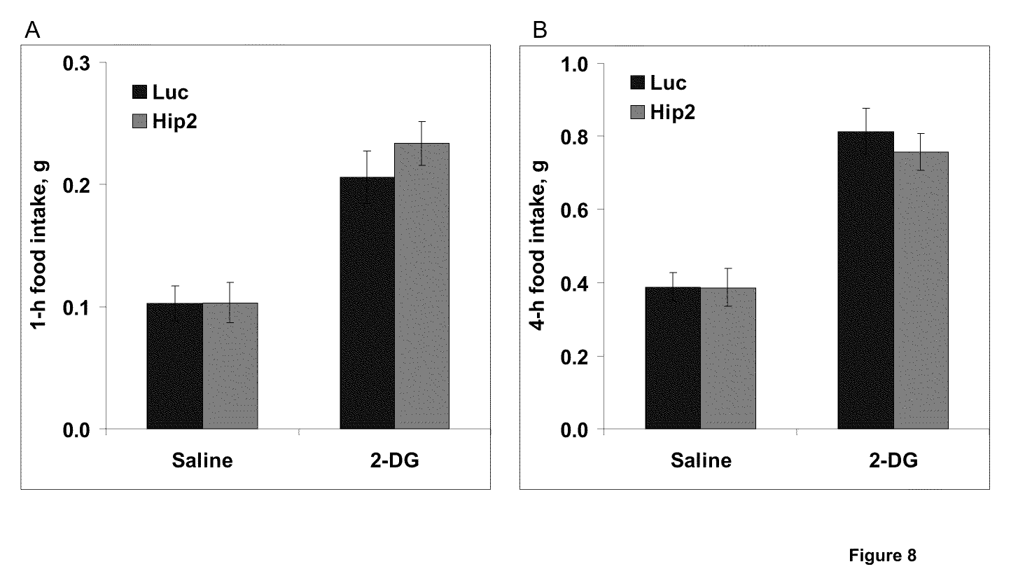

Silencing of Hip2 was achieved by AAV vectors encoding the Hip2 small hairpin RNA (Hip2 shRNA) delivered into the VMN region of the brain via injection. AAV vectors encoding the luciferase shRNA were used as controls. While reduction of Hip2 levels resulted in increased body weight as well as a more acute response in the animals ability to regulate and maintain core body temperature, Hip2 silencing did not alter the animals food intake at 3 and weeks (FIG. 6A-B) nor did it affect their daily physical activities (FIG. 7A-F). Furthermore, reduction of Hip2 levels also had no effect on the ability of the VMN neurons to sense glucose levels in blood. When mice were administered 2-deoxy-D-glucose (2DG) a non-metabolizable form of glucose that induces hyperphagia, there was no difference in the food intake between mice injected with the AAV vector for Hip2 shRNA versus mice injected with the vector for Luc shRNA (FIG. 8). These results show that Hip2 is not essential for the VMN neurons ability to sense blood glucose levels.

### Example 5

### Modulation of Neuronal Glucose Sensing by Estrogen Receptor Alpha (ERα)

Recent experiments have demonstrated that RNAi-mediated silencing of ERα in the neurons of the ventromedial nucleus (VMN) of the hypothalamus of adult mice leads to behavioral and physiological abnormalities closely resembling a metabolic syndrome. VMN contains populations of glucose-responsive (GR) neurons critical for the regulation of energy homeostasis by glucose. The majority of GR neurons use glucokinase (GK) as the key regulator of glucosensing. GK appears to modulate potassium (K)-ATP channel activity via its gatekeeper role in the glycolytic production of ATP. Consequently, GR neurons change their membrane potential and firing rate as a function of glucose concentration eventually initiating a counter regulatory response to elevated or reduced glucose levels in the blood.

It was hypothesized that ERα modulates the glucosensing function of GR neurons, thus integrating estrogen and glucose signaling pathways. The preliminary experiments have revealed that overexpression of ERα in a glucose-responsive murine hypothalamic cell line, N43, lead to upregulation of GK and Kir6.2, a pore-forming subunit of the K(ATP) channel. Concomitantly, the levels of glucose and lactose transporters were not affected. Furthermore, silencing of ERα in the VMN neurons of female mice suppressed the expression of both GK and Kir6.2. These results suggest an important role of ERα in glucosensing by hypothalamic neurons.

Furthermore, changes in ERα signaling in this brain region may be implicated in the development of the metabolic syndrome. Given that elevated levels of GK and Kir6.2 are expected to improve brain sensitivity to glucose, overexpression of ERα in the VMN neurons using viral vectors may thus hold promise as a novel approach to treat the metabolic syndrome in humans.

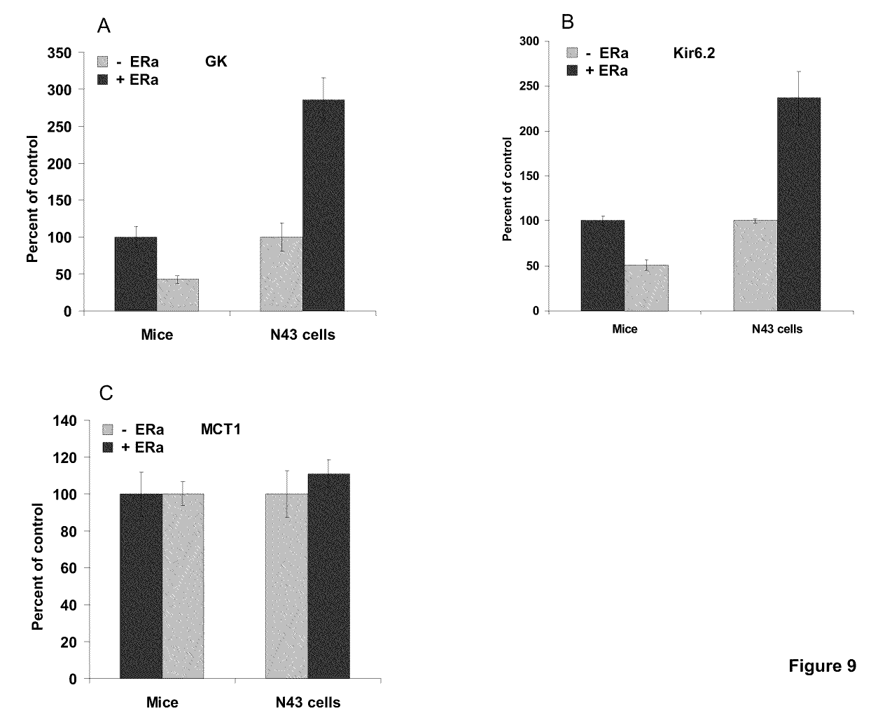

The effect ERα levels have on expression of genes essential for neuronal glucose sensing was determined. To address the role of endogenous ERα in glucose sensing by the VMN neurons, female mice were injected with AAV vectors encoding for small hairpin RNAs (shRNAs) targeting ERα or luciferase (negative control). Several weeks after surgery, brain regions containing VMN were dissected and the levels of GK, Kir6.2 and a lactose transporter MAT1 were analyzed by quantitative PCR, see FIGS. 9A, B and C respectively. Suppression of endogenous ERα resulted in downregulation of GK and Kir6.2 but not MAT1. Consistent with these results, overexpression of ERα in a murine hypothalamic N43 cell line using AAV vectors induced both GK and Kir6.2.

### Example 6

### AAV-Mediated Estrogen Receptor Alpha (ERα) Silencing in the VMN

AAV vectors were designed to express small hairpin RNA (shRNA) for silencing gene expression targeting murine ERα (GGCATGGAGCATCTCTACA, SEQ ID NO.:5) or firefly luciferase (CCGCTGGAGAGCAACTGCAT, SEQ ID NO.:6) under the control of the human H1 promoter. In addition, both vectors contained a second expression cassette for destabilized yellow fluorescent protein (YFP). The latter was used as a reporter to visualize transduced neurons. Vector stocks were generated using a helper-free AAV-2 plasmid transfection system, purified by heparin affinity chromatography and dialyzed against PBS. AAV genomic titers were determined by quantitative PCR and adjusted to 10⁹ particles per ml.

Stereotaxic surgery was performed to administer AAV vectors directly to the ventromedial nucleus (VMN) of the hypothalamus of adult mice and determined the effect ERα gene silencing had on tissues from the mice. All stereotaxic surgical procedures were performed on gonad-intact female mice (8-12 weeks old) under ketamine/xylazine anesthesia. Vectors (1 μl, 10⁹ particles) were injected into the VMN (AP−0.9, ML+/−0.6, DV−5.8) bilaterally over 10 min using a 10-ml Hamilton syringe and an infusion pump (World Precision Instruments). The needle was left inserted for an additional 5 min and then slowly withdrawn. Animals which were successfully targeted bilaterally and those with unilateral injections were analyzed as separate groups.

Approximately three weeks after surgery, female mice that were injected into the VMN with AAV vectors targeting luciferase (siLuc) or ERα (siER1) were sacrificed and perfused with 4% paraformaldehyde. The brains were analyzed by immunohistochemistry using a free floating section method and the following primary antibodies and stained for NeuN and ERα using anti-GFP (Abcam, ab290, 1:10,000), anti-ERα (Upstate Biotechnology, C1355, 1:10,000), and anti-NeuN (Chemicon, MAB377, 1:10,000). Accuracy of injections and the efficiency of ERα silencing were also assessed with YPF and ERα immunostaining. Animals with unilateral (siER1-U) and bilateral (siER1-B) injections were analyzed separately, p<0.05. It was noted that suppression of ERα expression by siER1 was restricted to the VMN.

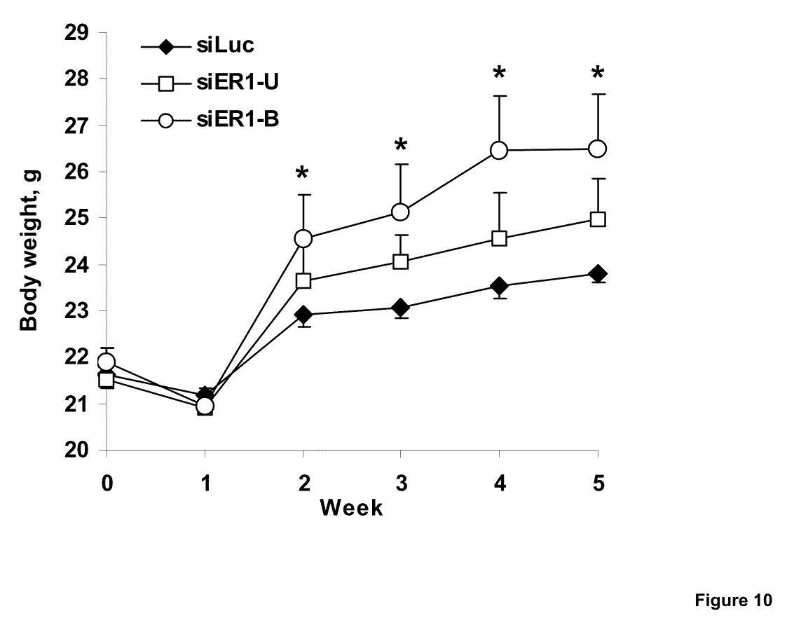

Prior to sacrificing the animals, the body weights of the mice injected with the indicated vectors were monitored over a period of 3 to 5 weeks post surgery. Statistically significant increases (p<0.05) in body weight were observed in siER1-B and siER1-U injected mice as opposed to marginal increases in body weight as seen in siLuc injected mice (FIG. 10).

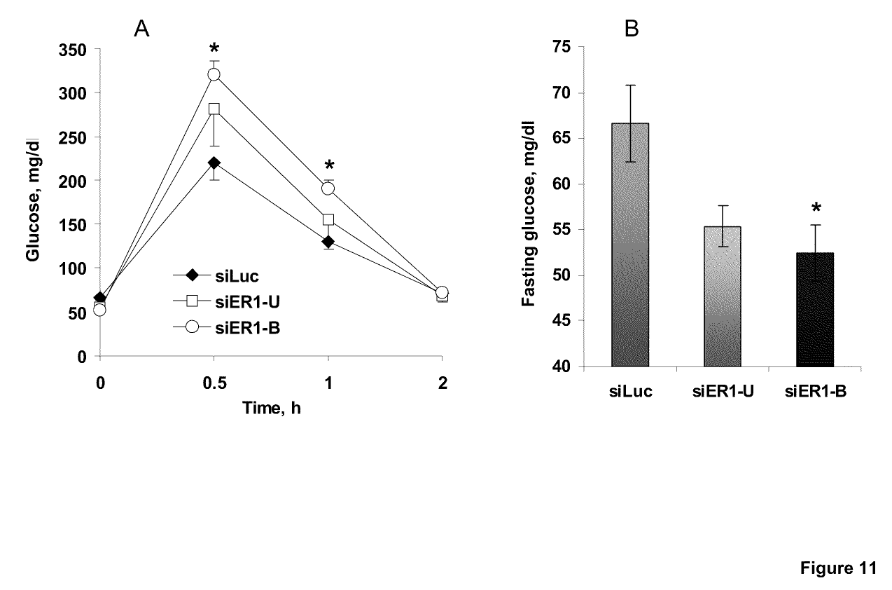

The effect ERα silencing had on fasting glucose levels was also investigated. Three weeks after surgery, glucose starved animals (performed during the dark phase of the daily cycle) were injected with glucose (2 mg/kg, i.p.) and monitored their blood glucose concentration for 2 h. Prior to the challenge, siER1-treated mice exhibited lower fasting glucose concentrations when compared to control animals (FIG. 11A). However after glucose challenge, higher levels of blood glucose in siER1-treated mice (p<0.05) (FIG. 11B) than controls were noted, indicating reduced glucose tolerance when ERα is silenced.

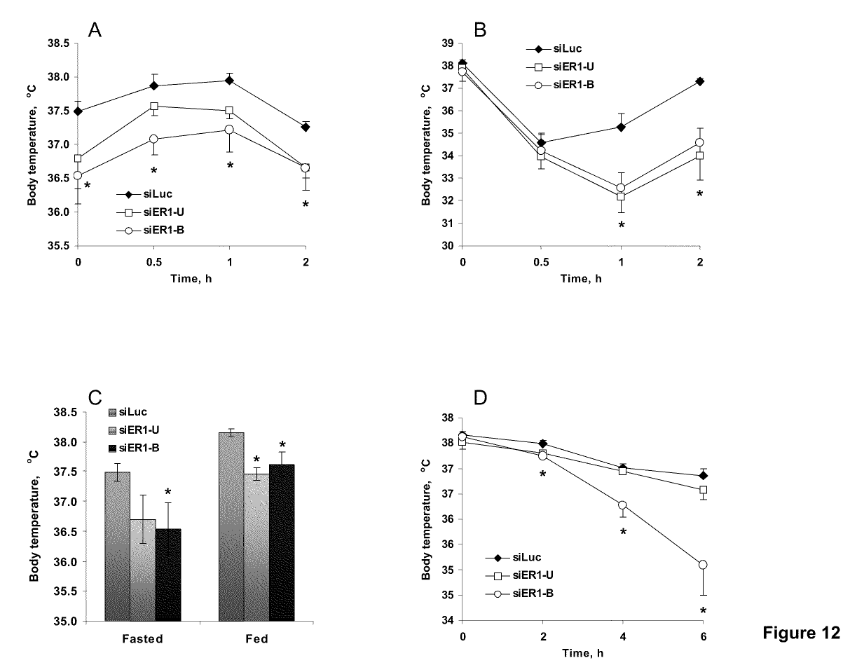

Core body temperature as an additional parameter during the glucose tolerance test described above was also measured. siER1-treated mice increased their body temperature after glucose injection. However, FIG. 12A illustrates that both baseline values and after glucose challenge values were significantly lower for siER1-treated mice when compared to control animals (p<0.05). Similar reductions in body temperature were also noted in siER1-treated mice when fasted and ad libitum fed (freely available), shown in FIG. 12B.

In another set of experiments, the effects of glucopenia and acute temperature stress had on body temperature was measured. Animals were systemically injected with 250 mg/kg of 2-deoxy-D-glucose (which is incapable of undergoing glycolysis) intraperitoneally and their body temperature was monitored for 2 h. Mice treated with siER1 vector developed a more profound and sustained hypothermia apparent in as little as 30 mins after induced glucopenia as compared to control animals, illustrated in FIG. 12C. Similarly in FIG. 12D, when mice were exposed to acute cold stress (4° C.), animals injected with siER1 displayed reduced ability to maintain body temperature.

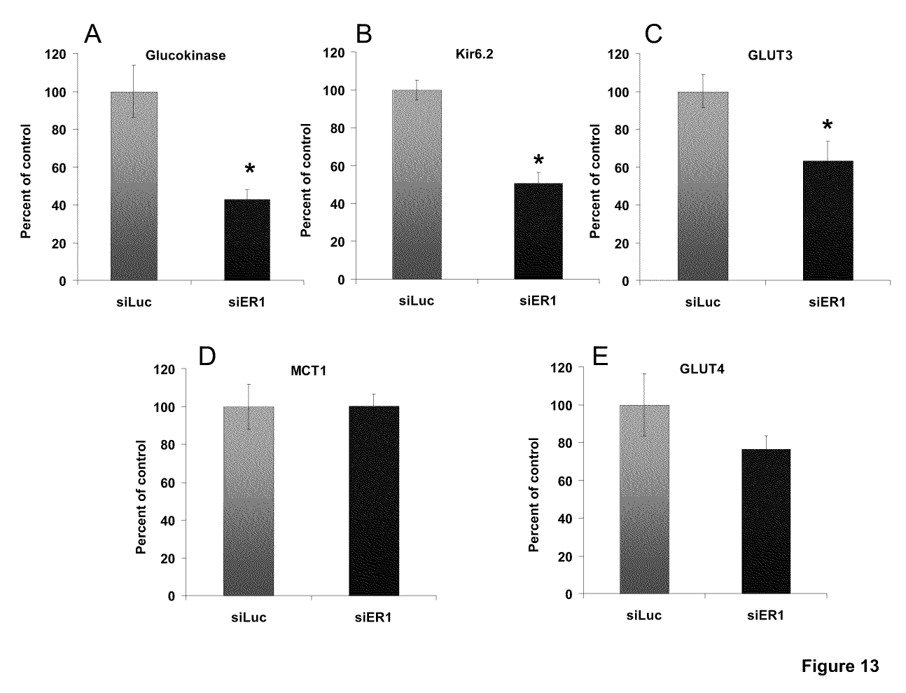

To better understand the role ERα plays in glucose metabolism, tissues from dissected VMN regions of injected mice were analyzed for expression of several genes implicated in neuronal glucosensing by quantitative PCR. Suppression of ERα in the VMA resulted in the significant reduction (p<0.05) in mRNA levels of several genes (FIG. 13A-F) including glucokinase (an enzyme that increases metabolism of glucose through phosphorylation), Kir6.2 (a pore-forming subunit of the K(ATP) channel) and glucose transporter GLUT3, as shown in FIG. 13B-C.

### Example 7

### AAV-Mediated Estrogen Receptor Alpha (ERα) Overexpression in Glucose-Responsive Cells In Vitro

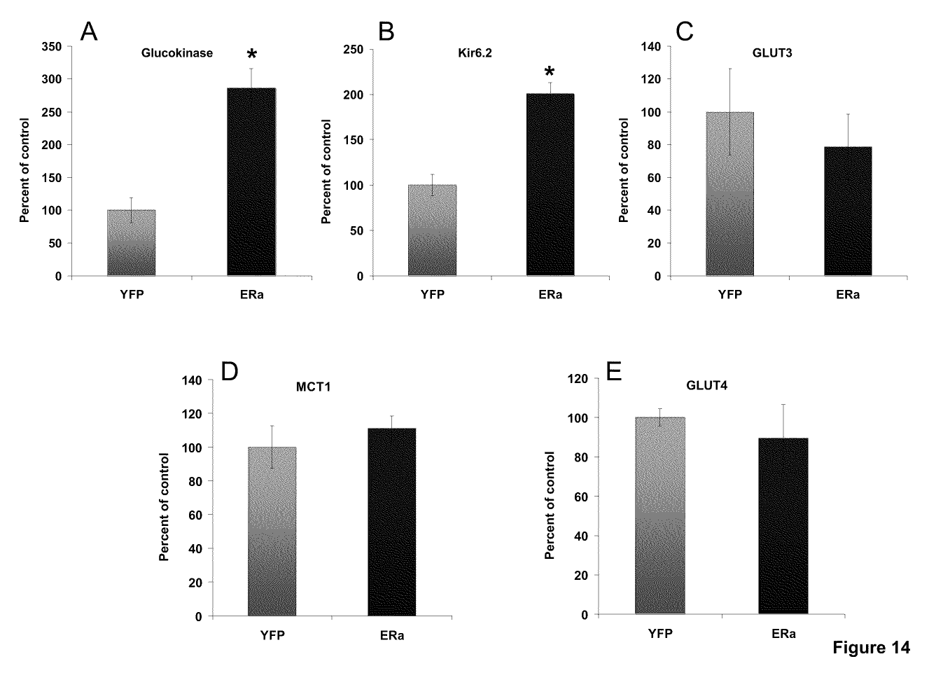

To determine if overexpression of ERα has the opposite effect as ERα silencing, ERα in a glucose sensitive cell line was overexpressed. Glucose-responsive murine hypothalamic N43 cells were transduced with over-expression ERα or YFP AAV vectors. Expression levels of glucosensing genes (FIG. 14A-F) were analyzed 48 h later by quantitative PCR and significant expression increases (p<0.05) were observed for glucokinase and Kir6.2, FIG. 14A-B.

### Example 8

### Expression of Membrane but not Nuclear Estrogen Receptor in Glucose Sensing Neurons of the Ventromedial Nucleus of the Hypothalamus

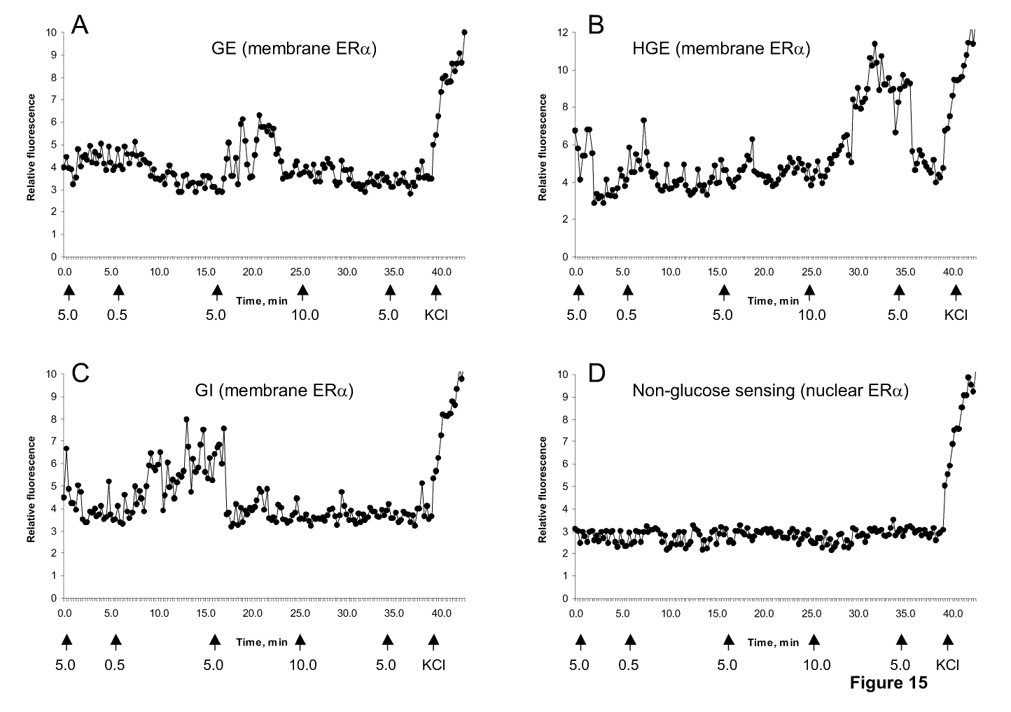

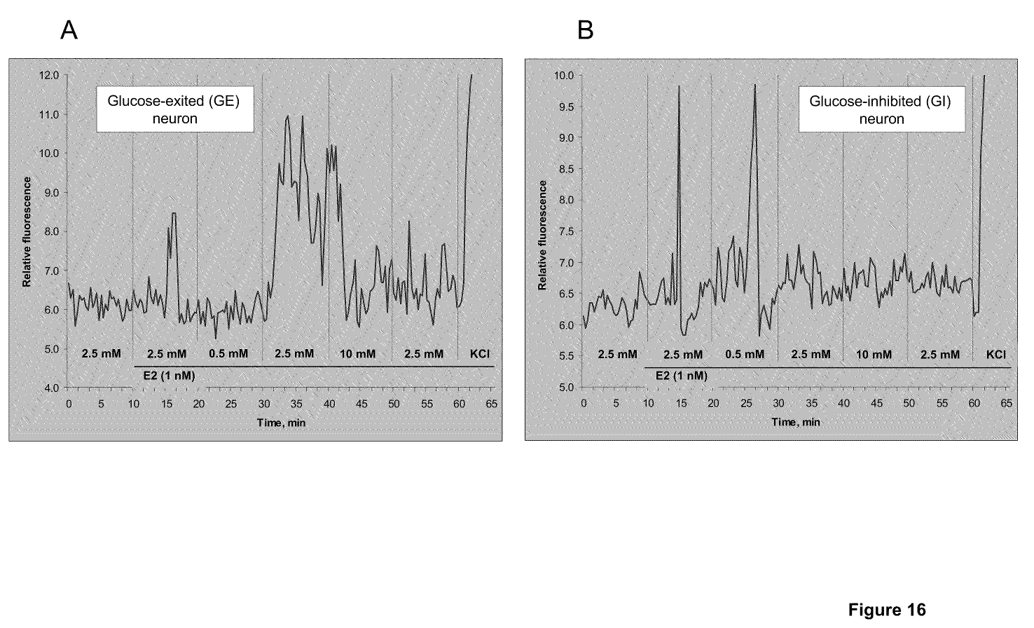

VMN contains specialized populations of glucose sensing neurons, which play a pivotal role in mediating a variety of physiological responses to changes in extracellular glucose levels. To address if ERα is expressed in glucose sensing neurons and modulates their function, thus integrating estrogen and glucose signaling pathways, changes in neuronal activity induced by glucose in dissociated VMN neurons from postnatal female mice were analyzed. The cells were perfused with varying concentrations of glucose and imaged using a membrane potential-sensitive dye or calcium followed by ERα immunocytochemistry. Surprisingly, none of the neurons that displayed robust ERα nuclear staining demonstrated any response to changes in glucose levels over a wide concentration range from 0.5 mM to 15 mM. Nonetheless, the majority of glucose-excited neurons were immunolabeled for ERα at the plasma membrane, see FIGS. 15A-D. Furthermore, unlike cells with nuclear ERα staining, a majority of membrane ERα-immunoreactive cells depolarized following application of 1 nM or 10 nM 17β-estradiol (FIG. 16A-B).

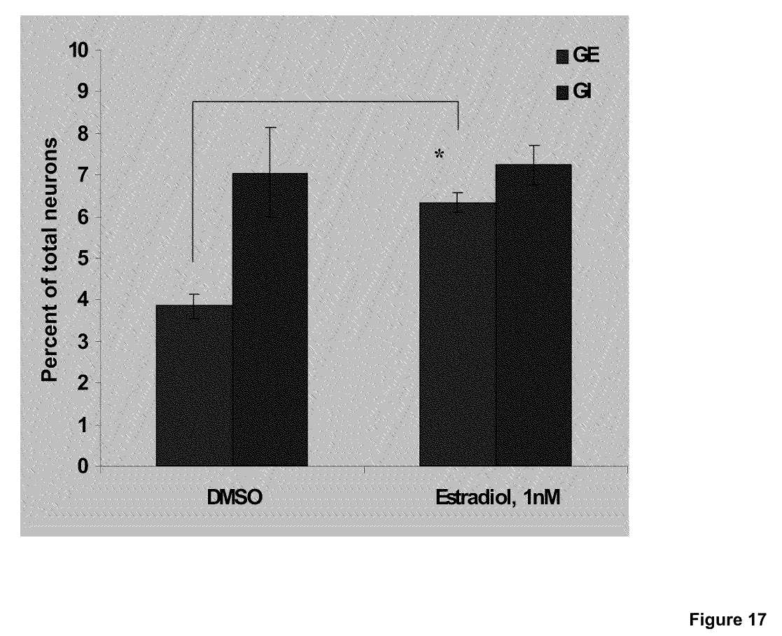

Overexpression of ERα in a murine hypothalamic cell line N43 (FIG. 9A-C) has led to upregulation of glucose sensing genes. In order to determine whether these effects are also dependent on estrogen, primary VMN neurons from female rats were treated 17-β-estradiol at physiological levels (1 nM) and the number of glucose sensing neurons were measured in 24 h using calcium imaging. It appears, that exposure to estrogen has increased the number of glucose-excited neurons (FIG. 17).

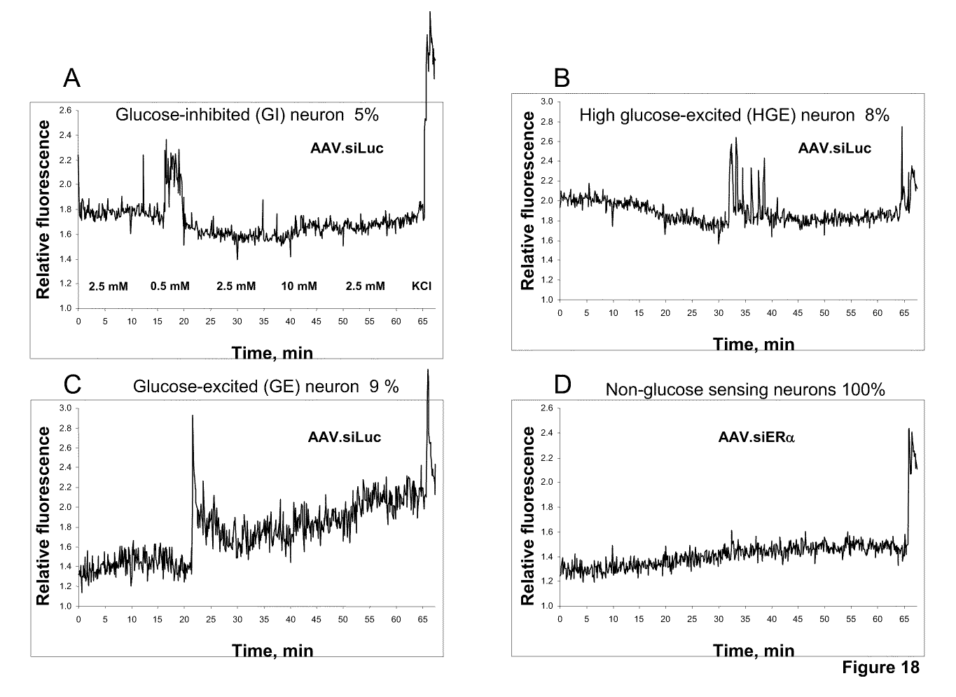

To directly address the significance of ERα in glucose sensing function, the receptor in cultured neurons was silenced using AAV-mediated RNA interference. While the total number of glucose sensing neurons decreased after 4 days in culture, the cells transduced with a control vector (siluc) contained subpopulations of all main types of glucose-responsive neurons including glucose-excited (GE)(FIG. 18A), glucose-inhibited (GI)(FIG. 18B) and high glucose-excited (HE)(FIG. 18C). In contrast, no cells transduced with a vector targeting ERα (siERα) (FIG. 18D) would respond to glucose in a wide range of concentrations. These findings suggest an involvement of membrane but not nuclear ERα signaling in the glucose sensing function of the neurons in the VMN. Given that the membrane form of the receptor is likely to be structurally distinct from the nuclear one, the membrane ERα holds a great promise as a therapeutic target for the treatment of at least certain aspects of metabolic syndrome that are likely to stem from impaired central glucose sensing, such as obesity and type II diabetes. The data are thus consistent with the in vivo experiments which identified ERα as a key regulator of neuronal glucose sensing in neurons.

While the present invention has been described in terms of specific methods, and compositions, it is understood that variations and modifications will occur to those skilled in the art upon consideration of the present invention. For example, the methods and compositions discussed herein can be utilized beyond treating metabolic disorders and pharmaceutical compositions in some embodiments. As well, the features illustrated or described in connection with one embodiment can be combined with the features of other embodiments. Such modifications and variations are intended to be included within the scope of the present invention. Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims.

All publications and references are herein expressly incorporated by reference in their entirety. The terms “a” and “an” can be used interchangeably, and are equivalent to the phrase “one or more” as utilized in the present application. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed:

1. A method for treating a metabolic disorder comprising:
identifying the metabolic disorder;
providing at least a portion of a gene to increase or decrease expression of a therapeutic protein;
incorporating the gene into a vector;
transfecting the vector into at least one cell in a central nervous system; and
increasing or decreasing expression of the therapeutic protein in at least one cell to thereby treat the metabolic disorder.

2. The method of claim 1, wherein the metabolic disorder is selected from the group consisting of obesity, type-2 diabetes, hypertension, wasting syndrome, cachexia and atherogenic dyslipidemia.

3. The method of claim 1, wherein the step of providing the gene comprises providing a polynucleotide sequence that functions as at least one of a shRNA, a siRNA, and a RNAi.

4. The method of claim 3, wherein the polynucleotide is homologous to at least a portion of an estrogen receptor-alpha (ERα) gene and decreases ERα protein expression.

5. The method of claim 4, wherein the step of transfecting the vector comprises delivering the vector to a glucose-responsive neuron of a ventromedial nucleus.

6. The method of claim 5 further comprises:
providing a second polynucleotide homologous to at least a portion of huntingtin interacting protein 2 (Hip2) gene to decrease expression of Hip2 protein;
incorporating the Hip2 polynucleotide into a vector;
transfecting the vector into at least one neuron in a hypothalamus; and
decreasing expression of the Hip2 protein in at least one neuron.

7. The method of claim 1, wherein the therapeutic protein is selected from the group consisting of huntingtin interacting protein 2 (Hip2), peroxisome proliferator-activated receptor γ coactivator 1α (PGC1-α), and estrogen receptor-alpha (ERα).

8. The method of claim 1, wherein the therapeutic protein is an estrogen receptor-alpha (ERα) protein.

9. The method of claim 1, wherein the gene is at least a portion of a gene from the group consisting of huntingtin interacting protein 2 (Hip2), peroxisome proliferator-activated receptor γ coactivator 1α (PGC1-α), and estrogen receptor-alpha (ERα).

10. The method of claim 9, wherein the gene is at least a portion of an ERα gene to increase expression of at least a portion of an ERα protein.

11. The method of claim 10 further comprises:
providing at least a portion of a Hip2 gene to increase expression of at least a portion of a Hip2 protein;
incorporating the Hip2 gene into a vector;
transfecting the vector into at least one neuron of the hypothalamus; and
increasing expression of the Hip2 protein in the at least one neuron.

12. The method of claim 1, wherein the step of incorporating the gene into the vector comprises incorporating the gene into a viral vector.

13. The method of claim 12, wherein the vector is selected from the group consisting of adeno-associated viral vector, herpes viral vector, parvoviral vector, and lentiviral vector.

14. The method of claim 12, wherein the viral vector is an adeno-associated viral vector (AAV).

15. The method of claim 14, wherein the AAV is a recombinant AAV having a cap-region from AAV type (1) and a rep-region from AAV type (2).

16. The method of claim 1, wherein the step of incorporating the gene into the vector comprises incorporating the gene into a non-viral vector.

17. The method of claim 16, wherein the non-viral vector is a liposome-mediated delivery vector.

18. The method of claim 1, wherein the step of transfecting the vector comprises delivering the vector to a desired region of the central nervous system using stereotaxic delivery.

19. The method of claim 1, wherein the step of transfecting the vector comprises delivering the vector to a desired region of a brain.

20. The method of claim 19, wherein the region of the brain is selected from the group consisting of hypothalamus, ventromedial nucleus, and arcuate nucleus.

21. The method of claim 1, wherein the step of transfecting the vector comprises delivering the vector to a ventromedial nucleus.

22. The method of claim 21, wherein the step of delivering the vector further comprises delivering the vector to a glucose-responsive neuron of the ventromedial nucleus.

23. A method for treating obesity comprising:
identifying a target site in a brain for modification of a patient in need thereof,
transfecting at least one cell at the target site with a vector expressing a therapeutic protein,
expressing the therapeutic protein in an amount effective for modulating metabolism in the patient.

24. The method of claim 23, wherein the target site of the brain is at least one of a hypothalamus, a ventromedial nucleus and an arcuate nucleus.

25. The method of claim 23, wherein the therapeutic protein is selected from a group consisting of brain derived neurotrophic factor (BDNF), huntingtin interacting protein 2 (Hip2), peroxisome proliferator-activated receptor γ coactivator 1α (PGC1-α), estrogen receptor-alpha (ERα), glial neurotrophic factor (GNF), erythropoietin (EPO), granulocyte colony stimulating factor (G-CSF), thrombopoietin (TPO), growth hormone (GH), interleukin 2 (IL-2), interferon-alpha receptor, interferon-beta receptor, and insulin receptor.

26. The method of claim 23, wherein the therapeutic protein is at least a portion of an estrogen receptor-alpha (ERα) protein.

27. The method of claim 26, wherein the cell is a glucose-responsive neuron of a ventromedial nucleus.

28. The method of claim 27 further comprises:
transfecting at least one neuron of the hypothalamus with a vector to express at least a portion of a huntingtin interacting protein 2 (Hip2) protein; and
expressing the Hip2 protein.

29. The method of claim 23, wherein the step of expressing the therapeutic protein comprises altering a basal metabolic rate to cause a reduction in body weight.

30. The method of claim 23, wherein the step of transfecting at least one cell comprises administering the vector by at least one of an oral administration, a nasal administration, a buccal administration, an intravenous injection, an intra-peritoneal injection, an intrathecal administration, and a route appropriate for delivering the vector to a particular region of the brain.

31. A pharmaceutical composition for treating a metabolic disorder comprising:
an effective amount of an adeno-associated viral vector encoding at least a portion of a gene to increase or decrease expression of a therapeutic protein in a desired region of a brain; and
a pharmaceutically acceptable carrier to treat the metabolic disorder.

32. The pharmaceutical composition of claim 31, wherein the disorder is selected from the group consisting of obesity, hypertension, diabetes, wasting syndrome, cachexia, and athrogenic dyslipidemia.

33. The pharmaceutical composition of claim 32, wherein the disorder is obesity.

34. The pharmaceutical composition of claim 31, wherein the at least the portion of the gene comprises an estrogen receptor-alpha (ERα) gene.

35. The pharmaceutical composition of claim 34 further comprising:
an effective amount of a vector comprising at least a portion of a huntingtin interacting protein 2 (Hip2) gene to increase expression of at least a portion of a Hip2 protein in a neuron of a hypothalamus.

36. The pharmaceutical composition of claim 31, wherein the gene comprises a polynucleotide sequence that functions as at least one of a shRNA, a siRNA and a RNAi to decrease expression of the therapeutic protein to therapeutically effective levels.

37. The pharmaceutical composition of claim 36, wherein the polynucleotide sequence is homologous to at least a portion of an estrogen receptor-alpha (ERα) gene.

38. The pharmaceutical composition of claim 37 further comprising:
an effective amount of a vector comprising a polynucleotide sequence that is homologous to at least a portion of a huntingtin interacting protein 2 (Hip2) gene to decrease expression of a Hip2 protein in a neuron of a hypothalamus.

39. The pharmaceutical composition of claim 31, wherein the therapeutic protein is selected from the group consisting of huntingtin interacting protein 2 (Hip2), brain derived neurotropic factor (BDNF), peroxisome proliferator-activated receptor γ coactivator 1α (PGC1-α), and estrogen receptor-alpha (ERα).

40. The pharmaceutical composition of claim 31, wherein the region of the brain is at least one of a hypothalamus, a ventromedial nucleus, and an arcuate nucleus.

41. The pharmaceutical composition of claim 31, wherein the therapeutic protein is functional in a ventromedial nucleus of a mammalian brain.

42. The pharmaceutical composition of claim 41, wherein the therapeutic protein is functional in a glucose-responsive neuron of the ventromedial nucleus.


SEQUENCE LISTING (6 sequences)

SEQ ID NO: 1 - DNA, 4866 bp, Mus musculus
agcgtggtgcgcatgcgcggacccggcgccattttggtggccgggcgcggaggtgattctacagtgaggaggaacgcgacggcgagggtggcagtggcagcgttcgtgtgctcgggtctgaatcgctgagggaggaggcggcggcggaggaagaggtggcagcggtggcggtggtcgtagcggtggcggaggaggcgggtacgaatcagctgcgggcagagacatggccaacatcgcggtgcagcgaatcaagcgggagttcaaggaggtgctgaagagcgaggagacgagcaaaaatcaaattaaagtagatcttgtagatgagaattttacagaattaagaggagaaatagcaggacctccagacacaccgtatgaaggtggaagatatcaactagagataaaaataccagaaacatatccatttaacccccctaaggtccggtttatcactaaaatatggcaccctaatattagttccgtcacaggggctatttgtttggatatcctgaaagatcaatgggcagcagcaatgactctgcgcacggtattattgtcattgcaagcgctgttggcggctgcagaaccagatgacccccaagatgcagtagtagcgaatcagtacaaacagaatcctgaaatgttcaagcagacagctcgactttgggcacacgtgtacgctggagcaccagtttctagtccagaatacaccaaaaaaatagaaaacctctgtgctatgggttttgataggaacgcagtaatagtggccttgtcttcaaaatcatgggatgtagagactgcaacagaactgcttctgagtaactgaggcacaggagctgcgccagcatcgctctcgcctcttgaggagcatcaacacctgttatttttaggattctgcataaagttcttttaaactggcattcttgcctaatgatgttacctaggcaccattggagactgcaaaaaaaaaaattccctgctctgtaaataaagctaattaaacgtctgtgtaaatttaaaaaggggaaatacttttaattttttttcttaatagtgtaaaaattctttgagctaagctaaaaccatgggaaaacatgctactttagtgtttagcagtgtaacagactcgagagtttgcttcagggttctgttgactaactaggataacactcgagtgtcggcccttcacattgtggtgtagaatcacagctcccttaagtctgaacacagatcctctgtgcctgtgaatttacttgcatgcttgtacttgacttcttagaacgggtagccgaaaagaccacccttttaagcattaggacattcttgaattatgagatttatcctgatttaaaggtggtgacctaatcagagctttttagtccctgtcaccatccagagcggagattctcatggtcaatttattttgtgtcttgtgggaaacgtgctgaggaacagtgtcccttgttccacttttagctcataggtttggaatgttttgtccgagttcttcactttgtaagtagtaatgtaaaaaggggctgccaattttatttctctcacaaaaggaataaatacttgatgttgcagtatttctagatttaataaaaggacactgcttagtttttcagtgaatttgacattaatttttaaaatgataaagtctttggaaacttgaagggtgcagtacctgttccctagtgctcagcctgacagtttcctttgtcagacacttgacttactcctcctgctccccatggtgacccatgaaagctgtgctgtctcactgtatataccagatgcacataggaacaggggtgtattctaaatctagctttctttatgatgtttctgataatgtgagaattgaaactttaccttctcttgtacataaacttcctgtccttagcaactgtcgcatctcattctatcttccaaagcttgacaattataagttttacaaactcaaatacttcctttaaccatcctaattaatgttcaagtgaacttaggcagttgttacataggagatacaattaaaaattttgaggaccaatgcatagattacagaaatgtaatggaatcagactaaattaaagtaaggttgtatgtatattgaaagtcatattctaaaaggtttgttttcttgaagtattgtttcaccttacagtcatgaagtgtttcaaaaatactttttgaacctattatctcagcataacttcactttacttttaataatcttgatactacaagcaataaaagaagcattaatatattataacattcaagatgtgacggctcttccagcgacttgtaaggctgtcacagttctcagtgactgagggccactgtcctccacactaaactcaagctgctatgtttttctccatgtttgcggatgcacacatttcctgttgcagctacatccttttattgctgtaggttattttataaagatgccaaaggaaacaagtttttctttatttattttaagtagctaaaactttgtactgttctttatattctcagataagaaggaaatgtgtgaaataaccatgagtttacctccatagttcattagaacttttaatggagggtggcttggtaacactaatatttaggtattggaaagtataacacatgttcttggagtttactctctgcctgtattgtcagggtacattgtctagtagatgagagctcagacaacaaaagtcaggcagtacctactttgtcttggtggcagctgtgttctttttttgttttttgtttttttgtttttttgtttttttgcatattggcttcagactggctccactgaatacttactggagatgtatatataacatgtggtttcatagatttgacgtgattatgccatcgcctcctgtcagaaccgtgtgctcaagactgtgtgggattcataagcaagactgcttggggtttctcatgttcatttaatgtgctaatcacccttttggggtattttccctcacacttctcgtttgttttttatttttcttttgtttcccaactcgtaggtttagaaagaagcagcttggtccttatcctggtgtttcatacagttaggaaaacaatgcgaaaaattaagatttagtcaaaagtaccctgatggaaattattttttttagtctgcgtttgacagtttttcattttaacaagtactacacaggttctgttttggtgtaggtgtgatggcgcttttactaccaggttcttgaaagagaaaggcaggcagagtctgtgagaggtgtatgcccagtcttagccccctgttcccataattttagtgaaatcgcgtttagagcaggctgactccctcatcctcgctgatcctctgatcagcttttctttctgagcctaggcctgacttttagtataaaacttagagagcatgttgggaacttgggattcaaaaaacatgtcagtgtttggaattgaatattttacctttgagaagtgtaactaacctatcatatgaattaaaccatcctttttgaaatttttttttatatttctgattgttggtggcatttaacactatgcaaaaaacttaagtatctgcagaaaggaactgacagtaggttttgctcacgcggttagacattttgcacagcgtgttatacgttactggttaggctgttgggccagtttacagaggttaggatgttgggtaagtggagcccggatgtgattgtaggtctcatacagcttttaaaactgcaggagaagtagatgttcttgcagcctactgtagcagcaccggaaatgacgttttggtggggtaagggtttgaaatgaatttgggaattgagcagtggagtcaggtatattgtataaagatgggatcagtgtttagataagaaaaaggtaggaaggtgtctggtcatagttactttatttcatggactttgataagatagttctgtattaatcactttttctagtttcaaggagttgatgttggaagttacattttcatctttcagtacctgtcttagtgtgtacccatcagcctgttggtgtcactaagggatagccttttctcaagcaagacttccagtgtctacacatgttgtgacaaacctacatacagcataaggtagcgggtgatgttggtgtctccttacctatcttgtgactgcttccttagactgctagaaactccggcgcctgcagtcatcactggaaggtgtcatgattgtgtcgcctttggcgtagttatccccccaccccacccctagccgtctatgatgaggtgtggccaggtctgtttggttctgaggcaagtggatcaggtcatagcactgctttttctgatcgtgtctcctattgtcacctactttttacaggcatcataacaaaagctagacaatttctttgggggaaaaaaaagacttatttaatgtaatttaaagacttttccaatagaaaatgaaatattattgaaaatgatatctattttttagctttcagcatttgatggtgctttgtcgtggtgtctgctggaagttactgccattatagggattttcattaacctcactttgaaagaaccttgcttgttggcttctttagatcttaaacagtttgttagtgatggtaattctgtaggagggtctattctgagccattaacttcttgtaaggggaaaatgggctggttaccagaaatattgaagcagggtgggctgtgggtggggggttgggtgtctgtcttgagaattaaaaactacgaaacacttttgtatacaactgaatttttaaaaaataaacacatttttaaagatgttgaattttccccattattgggaattcttaaaaataaatgcatgcacattccccctgc

SEQ ID NO: 2 - protein, 200 aa, Mus musculus
MANIAVQRIKREFKEVLKSEETSKNQIKVDLVDENFTELRGEIAGPPDTPYEGGRYQLEIKIPETYPFNPPKVRFITKIWHPNISSVTGAICLDILKDQWAAAMTLRTVLLSLQALLAAAEPDDPQDAVVANQYKQNPEMFKQTARLWAHVYAGAPVSSPEYTKKIENLCAMGFDRNAVIVALSSKSWDVETATELLLSN

SEQ ID NO: 3 - DNA, 4319 bp, Mus musculus
acaaggctccctgctcagcagccttgtgatgccaggagaggccaatgccccctcctctgccattgtctagcttaagaaggcagtctctgggcgacattcttctcaagcaggtggcccacgcgctgctgagccctctgcgtgcgcggggagccagtctgtaactcgccggctgccacttaccatgaccatgacccttcacaccaaagcctcgggaatggccttgctgcaccagatccaagggaacgagctggagcccctcaaccgcccgcagctcaagatgcccatggagagggccctgggcgaggtatacgtggacaacagcaagcccactgtgttcaactaccccgagggcgccgcctacgagttcaacgccgccgccgccgccgccgccgccgcctcggcgccggtctacggccagtcgggcatcgcctacggccccgggtcggaggcggccgccttcagtgccaacagcctgggggctttcccccagctcaacagcgtgtcgcctagcccgctgatgctgctgcacccgccgccgcagctgtctcctttcctgcacccgcacggccagcaggtgccctactacctggagaacgagcccagcgcctacgccgtgcgcgacaccggccctcccgccttctacaggtctaattctgacaatcgacgccagaatggccgagagagactgtccagcagtaacgagaaaggaaacatgatcatggagtctgccaaggagactcgctactgtgccgtgtgcaatgactatgcctctggctaccattatggggtctggtcctgcgaaggctgcaaggctttctttaagagaagcattcaaggacacaatgactacatgtgtccagctacaaaccaatgcaccattgacaagaaccggaggaagagttgccaggcctgtcggctgcgcaagtgttacgaagtgggcatgatgaaaggcggcatacggaaagaccgccgaggagggagaatgttgaagcacaagcgtcagagagatgacttggaaggccgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgctggctacgtcaagtcggttccgcatgatgaacctgcagggagaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcacatgagtaacaaaggcatggagcatctctacaacatgaaatgcaagaacgttgtgcccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctgagagctcccaggctccccgaaaggttctgagaatccctgcaacattttacccatgtcatgtatgactagcagaattctgtctcctgcgtacactgtggcatgcatccaccaccagtggctttctagtatgagtggccatttatttgcttgctcagttcttagtggcatgtcttctgttgggaacaggcaaaagggattccagggctaaatcttggtaacagctctctttctccctttgctacgtcactaactatgaggattcctatagtcttcagtcttcatgaatgaattgagataactgcattgaagctacttgttgagacccaagcctagagagtaaacatttcacctctgataagcacttcataatggctccaagaggaagcctcagcaaagacttcaaagtggctcctttaattgaagtcgttgacaaagcgatctcatggcttcctaatagcacccgcttgcattcctatagcaacatgtgactttattaaagggaaatcttgagcccctataggaatatgtgatgcacacagaaaaggaagatgcccccagttgtcaagaccatgtcaactcaatacagtcaaccttttcacttcttaaagtgtcagctgccgagtattatatacatggtgggttctagacaattcttgcttcccatgacttgtgggaaacagaaagtctatttcagttaataccctggaatgaattagtctccttcatgagggctgtagggtgtgttgattttaatttgatttcattttataatagtcttctgcccctggggcttgcagtaaaggcagcttgaggacctgttctggtaaactggacattcttcagcacaggatgctagccttgtctcttccctgatgtcaattgccctaaattgttaccatgtagattgatcaagggcagtcacaatgaacctgcaagctggtgacaattttttggtttgtttagttttattcttgggggcagggaacaagtaatgtatgggcaatgactgtgactcggaggagtctaagaaggaaagtaagggagaaatgtggagaatgacctggccagggcagcagcttcccagaggtgcacctcacagatcccaccatgcacagtgaggctgctctgggatccatgggctgacttcacttacatttcaggtatgagcctgattggaagaggtgacatcagatacctgtgttcctgtagatgctccaggaacttggggaaaggcatgggggcaagatcttggagcactcatgactcaagctgttgagaagttgctcagactagactgtgcttgattattctgaactcatccaaaaaccagctagtgagatcttggggagaaaaatttcttcctcaacacagttcatgatcttaaattttgtaaactagaaatgatgaatgaccagaattgggtcatgagagtcctttgaacaaggggataaaaaggaagacttactctatccacagccacccaagacctctggggtcttggtctctctagtacaactaaggattacttccttttccaaaccaaaatgaaagttcagtttccatcccggacttcatgatctataatttgaactccattgagatatgatgggttagccaaaacctaggtgaatggctaggcttctcttggcctgtactttttggaatggtgggttccattaatttctgctctcccagttaactgactatcaaagatgtggaaactgggagagccaatttttcttcttattttttttaaatttctcatacttgttcatcaaattcaaggacaattttatgtatatgctttaagaacatgtttaagaacctaactcttttgtttgtgtttaagaagcaccttatatagtataatatatatttttgttgaagtttaattgcttgtttattggacaattgaatgtaatagttatgttctgagttttaatttgattgggtttaacactgcaaaagccaaggggaaaatatttagtttgttttttttttttttttttgtagacgtttcaaactaccgtgtcatgtatgcagttattaatgcctaaagccttgtgattttcatttaaatgaagatcacatttcgtatcaacttttgtatccacagtagacaaaatagaaggaatccacatgcctattgctgggtgttgaatgacagacaatcttatgtagcagagattatgcctgggaaggaaaattattcagggcagctaattttgcttttaccaaaatatctgtagtaatatttttggacagtagctaatgggtcagtgggttctttttaatgtttatactgagatttttcttttacaaaaataaaaacaaaaatt

SEQ ID NO: 4 - protein, 599 aa, Mus musculus
MTMTLHTKASGMALLHQIQGNELEPLNRPQLKMPMERALGEVYVDNSKPTVFNYPEGAAYEFNAAAAAAAAASAPVYGQSGIAYGPGSEAAAFSANSLGAFPQLNSVSPSPLMLLHPPPQLSPFLHPHGQQVPYYLENEPSAYAVRDTGPPAFYRSNSDNRRQNGRERLSSSNEKGNMIMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDLEGRNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKGMEHLYNMKCKNVVPLYDLLLEMLDAHRLHAPASRMGVPPEEPSQTQLATTSSTSAHSLQTYYIPPEAEGFPNTI

SEQ ID NO: 5 - DNA, 19 bp, Mus musculus
ggcatggagcatctctaca

SEQ ID NO: 6 - DNA, 20 bp, Photinus pyralis
ccgctggagagcaactgcat